(19) United States
(12) Patent Application Publication
(10) Pub. No.: US 2025/0018039 A1
(43) Pub. Date: Jan. 16, 2025

(54) HLA-A3-RESTRICTED T CELL RECEPTORS AGAINST RAS WITH G12V MUTATION

(71) Applicant: The United States of America, as represented by the Secretary, Department of Health, Bethesda, MD (US)

(72) Inventors: Zhiya Yu, Bethesda, MD (US); Catherine M. Ade, Fairfax, VA (US); Matthew J. Sporn, New Rochelle, NY (US); James C. Yang, Bethesda, MD (US); Ken-ichi Hanada, Bethesda, MD (US)

(73) Assignee: The United States of America, as represented by the Secretary, Department of Health, Bethesda, MD (US)

(21) Appl. No.: 18/713,490

(22) PCT Filed: Nov. 30, 2022
(86) PCT No.: PCT/US2022/080650

§ 371 Date: May 24, 2024

Related U.S. Application Data
(60) Provisional application No. 63/284,884, filed on Dec. 1, 2021.

(51) Int. Cl. A61K 39/00; A61P 35/00; C07K 14/725; C07K 16/40

(52) U.S. Cl. CPC A61K 39/464464; A61K 39/4611; A61K 39/4632; A61P 35/00; C07K 14/7051; C07K 16/40

(57) ABSTRACT

Disclosed is an isolated or purified T cell receptor (TCR), wherein the TCR has antigenic specificity for a mutated human RAS amino acid sequence with a substitution of glycine at position 12 with valine. The TCRs may recognize G12V RAS presented by HLA-A3. Related poly peptides and proteins, as well as related nucleic acids, recombinant expression vectors, host cells, populations of cells, and pharmaceutical compositions are also provided. Also disclosed are methods of detecting the presence of cancer in a mammal and methods of treating or preventing cancer in a mammal.

### CROSS-REFERENCE TO RELATED APPLICATIONS

This patent application claims the benefit of U.S. Provisional Patent Application No. 63/284,884, filed Dec. 1, 2021, which is incorporated by reference in its entirety herein.

### STATEMENT REGARDING FEDERALLY SPONSORED RESEARCH OR DEVELOPMENT

This invention was made with Government support under project number ZIA BC 011843 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 44,325 Byte XML file named “764810.XML,” dated Nov. 29, 2022.

### BACKGROUND OF THE INVENTION

Some cancers may have very limited treatment options, particularly when the cancer becomes metastatic and unresectable. Despite advances in treatments such as, for example, surgery, chemotherapy, and radiation therapy, the prognosis for many cancers, such as, for example, pancreatic, colorectal, lung, endometrial, ovarian, and prostate cancers, may be poor. Accordingly, there exists an unmet need for additional treatments for cancer.

### BRIEF SUMMARY OF THE INVENTION

An aspect of the invention provides an isolated or purified T-cell receptor (TCR) comprising the amino acid sequences of: (a) all of SEQ ID NOs: 1-3, (b) all of SEQ ID NOs: 4-6, or (c) all of SEQ ID NOs: 1-6, wherein the TCR has antigenic specificity for a mutated human RAS amino acid sequence with a substitution of glycine at position 12 with valine, wherein the mutated human RAS amino acid sequence is a mutated human Kirsten rat sarcoma viral oncogene homolog (KRAS), a mutated human Harvey rat sarcoma viral oncogene homolog (HRAS), or a mutated human Neuroblastoma rat sarcoma viral oncogene homolog (NRAS) amino acid sequence, and wherein position 12 is defined by reference to the wild-type human KRAS, wild-type human HRAS, or wild-type human NRAS protein, respectively.

Another aspect of the invention provides an isolated or purified polypeptide comprising a functional portion of the inventive TCR, wherein the functional portion comprises the amino acid sequences of: (a) all of SEQ ID NOs: 1-3, (b) all of SEQ ID NOs: 4-6, or (c) all of SEQ ID NOs: 1-6.

Still another aspect of the invention provides an isolated or purified protein comprising at least one of the inventive polypeptides.

Another aspect of the invention provides a bispecific engager TCR fusion protein comprising (i) any of the inventive TCRs, polypeptides, or proteins described herein and (ii) an anti-CD3 engager.

Aspects of the invention further provide nucleic acids, recombinant expression vectors, host cells, populations of cells, and pharmaceutical compositions relating to the inventive TCRs, polypeptides, and proteins.

Another aspect of the invention provides an isolated or purified nucleic acid comprising, from 5′ to 3′, a first nucleic acid sequence and a second nucleotide sequence, wherein the first and second nucleotide sequence, respectively, encode the amino sequences of SEQ ID NOs: 7 and 8; 8 and 7; 9 and 10; 10 and 9; 23 and 24; 24 and 23; 25 and 26; 26 and 25; 27 and 28; 28 and 27; 29 and 30; or 30 and 29.

Methods of detecting the presence of cancer in a mammal, methods of treating or preventing cancer in a mammal, methods of inducing an immune response against a cancer in a mammal, methods of producing a host cell expressing a TCR that has antigenic specificity for the peptide of VVVGAVGVGK (SEQ ID NO: 33), and methods of producing the inventive TCRs, polypeptides, and proteins, are further provided by aspects of the invention.

### BRIEF DESCRIPTION OF THE SEVERAL VIEWS OF THE DRAWING(S)

FIG. 1 is a graph showing the average tumor size (mm²) measured in tumor-bearing mice at the indicated number of days following adoptive cell therapy (ACT) with human T cells transduced with the retroviral vector of Example 2 (mTCR), phosphate-buffered saline (PBS) (control), or human T cells transduced with an empty vector (mock).

FIGS. 2A-2B are graphs showing the levels of IFN-gamma (pg/mL) secreted following co-culture of target cells with effector cells. Target cells were pulsed with the RG12V RAS peptide VVVGAVGVGK (SEQ ID NO: 33) (2A) (mutant peptide titration) or the corresponding wild-type peptide VVVGAGGVGK (WT) (SEQ ID NO: 34) (2B) (wild-type peptide titration) at the concentrations shown. Effector cells were PBL from healthy human donors transduced with a retroviral vector encoding TCR 1 or TCR 807. PBL transduced with an empty vector (mock) served as a control.

FIGS. 3A-3B are graphs showing the levels of IFN-gamma (pg/mL) secreted following co-culture of target cells with effector cells. Target cells included (i) Cos cells transfected with HLA-A3 alone or both HLA-A3 and G12V RAS (FIG. 3A); (ii) RAS G12V positive tumor cell lines CFPAC, NCI H441, PACA, SW480, or SW620 that were either untransfected or transfected with HLA-A3, (FIG. 3A); or (iii) genetically unmodified patient-derived xenograft (PDX) tumor cell lines (4393, 4385, 4316, 4395, or 4391) (FIG. 3B). Effector cells were as described for FIGS. 2A-2B. The symbol “+” indicates that the target cell is positive for expression of HLA-A3 or RAS G12V. The symbol “−” indicates that the target cell is negative for expression of HLA-A3 or RAS G12V.

FIGS. 4A-4B are graphs showing the integrated intensity per image (normalized to 0 hours) (%) measured (with INCUCYTE live cell analysis system, Sartorius Corporation, Bohemia, NY) over a 48-hour co-culture of target cells with effector cells. HLA-A*03:01+/G12V KRAS+ target cells were tumor cell line 4391 (A) or tumor cell line 4316 (B). Effector cells were PBL from healthy human donors transduced with a retroviral vector encoding TCR 807 (circles) or TCR 1 (squares). PBL transduced with an empty vector (mock) (triangles) served as a control.

### DETAILED DESCRIPTION OF THE INVENTION

RAS family proteins belong to the large family of small GTPases. Without being bound to a particular theory or mechanism, it is believed that, when mutated, RAS proteins may be involved in signal transduction early in the oncogenesis of many human cancers. A single amino acid substitution may activate the protein. The mutated RAS protein product may be constitutively activated. Mutated RAS proteins may be expressed in any of a variety of human cancers such as, for example, pancreatic (e.g., pancreatic carcinoma), colorectal, lung (e.g., lung adenocarcinoma), endometrial, ovarian (e.g., epithelial ovarian cancer), and prostate cancers. The human RAS family proteins include KRAS, HRAS, and NRAS.

KRAS is also referred to as GTPase KRas, V-Ki-Ras2 Kirsten rat sarcoma viral oncogene, or KRAS2. There are two transcript variants of KRAS: KRAS variant A and KRAS variant B. Wild-type (WT) KRAS variant A has the amino acid sequence of SEQ ID NO: 11. WT KRAS variant B has the amino acid sequence of SEQ ID NO: 12. Hereinafter, references to “KRAS” (mutated or unmutated (WT)) refer to both variant A and variant B. unless specified otherwise. When activated, mutated KRAS binds to guanosine-5′-triphosphate (GTP) and converts GTP to guanosine 5′-diphosphate (GDP).

HRAS is another member of the RAS protein family. HRAS is also referred to as Harvey Rat Sarcoma Viral Oncoprotein, V-Ha-Ras Harvey Rat Sarcoma Viral Oncogene Homolog, or Ras Family Small GTP Binding Protein H-Ras. WT HRAS has the amino acid sequence of SEQ ID NO: 13.

NRAS is still another member of the RAS protein family. NRAS is also referred to as GTPase NRas, V-Ras Neuroblastoma RAS Viral Oncogene Homolog, or NRAS1. WT NRAS has the amino acid sequence of SEQ ID NO: 14.

An aspect of the invention provides an isolated or purified TCR, wherein the TCR has antigenic specificity for a mutated human RAS amino acid sequence with a substitution of glycine at position 12 with valine, wherein the mutated human RAS amino acid sequence is a mutated human KRAS, a mutated human HRAS, or a mutated human NRAS amino acid sequence, and wherein position 12 is defined by reference to the WT human KRAS, WT human HRAS, or WT human NRAS protein, respectively. Hereinafter, references to a “TCR” also refer to functional portions and functional variants of the TCR, unless specified otherwise.

The mutated human RAS amino acid sequence may be a mutated human KRAS amino acid sequence, a mutated human HRAS amino acid sequence, or a mutated human NRAS amino acid sequence. The amino acid sequences of WT human KRAS, NRAS, and HRAS protein each have a length of 188 or 189 amino acid residues and have a high degree of identity to one another. For example, the amino acid sequence of the WT human NRAS protein is 86.8% identical to that of the WT human KRAS protein. Amino acid residues 1-86 of the WT human NRAS protein and the WT human KRAS protein are 100% identical. The amino acid sequence of the WT human HRAS protein is 86.3% identical to that of the WT human KRAS protein. Amino acid residues 1-94 of the WT human HRAS protein and the WT human KRAS protein are 100% identical. Hereinafter, references to “RAS” (mutated or unmutated (WT)) collectively refer to KRAS, HRAS, and NRAS, unless specified otherwise.

In an aspect of the invention, the mutated human RAS amino acid sequence comprises a human RAS amino acid sequence with a substitution of glycine at position 12 with valine, wherein position 12 is defined by reference to the corresponding WT RAS protein. The WT RAS protein may be any one of WT KRAS protein (SEQ ID NO: 11 or 12), WT HRAS protein (SEQ ID NO: 13), or WT NRAS protein (SEQ ID NO: 14) because, as explained above, amino acid residues 1-86 of the WT human NRAS protein and the WT human KRAS protein are 100% identical, and amino acid residues 1-94 of the WT human HRAS protein and the WT human KRAS protein are 100% identical. Accordingly, the amino acid residue at position 12 of each of WT KRAS, WT HRAS, and WT NRAS protein is the same, namely, glycine.

The mutated human RAS amino acid sequence has a substitution of glycine at position 12 with valine. In this regard, aspects of the invention provide TCRs with antigenic specificity for any human RAS protein, polypeptide or peptide amino acid sequence with a G12V mutation.

Mutations and substitutions of RAS are defined herein by reference to the amino acid sequence of the corresponding WT RAS protein. Thus, mutations and substitutions of RAS are described herein by reference to the amino acid residue present at a particular position in WT RAS protein (namely, position 12), followed by the position number, followed by the amino acid residue with which that residue has been replaced in the particular mutation or substitution under discussion. A RAS amino acid sequence (e.g., a RAS peptide) may comprise fewer than all of the amino acid residues of the full-length, WT RAS protein. Accordingly, position 12 is defined herein by reference to the WT full-length RAS protein (namely, any one of SEQ ID NOs: 11-14) with the understanding that the actual position of the corresponding residue in a particular example of a RAS amino acid sequence may be different. When the positions are as defined by any one of SEQ ID NOs: 11-14, the term “G12” refers to the glycine normally present at position 12 of any one of SEQ ID NOs: 11-14, and “G12V” indicates that the glycine normally present at position 12 of any one of SEQ ID NOs: 11-14 is replaced by valine. For example, when a particular example of a RAS amino acid sequence is, e.g., VVVGAGGVGK (SEQ ID NO: 34) (an exemplary WT KRAS peptide corresponding to contiguous amino acid residues 7 to 16 of SEQ ID NO: 11), “G12V” refers to a substitution of the underlined glycine in SEQ ID NO: 34 with valine, even though the actual position of the underlined glycine in SEQ ID NO: 34 is 6. Human RAS amino acid sequences with the G12V mutation are hereinafter referred to as “G12V RAS.”

Examples of full-length RAS proteins with the G12V mutation are set forth in Table 1 below.

TABLE 1

| Mutated Full-Length RAS Protein | SEQ ID NO: |
|---|---|
| G12V KRAS variant A | 15 |
| G12V KRAS variant B | 16 |
| G12V HRAS | 17 |
| G12V NRAS | 18 |

In an aspect of the invention, the TCRs have antigenic specificity for a RAS peptide with the G12V mutation described above, wherein the mutated RAS peptide has any length suitable for binding to any of the HLA-A3 molecules described herein. For example, the TCRs may have antigenic specificity for a RAS peptide with the G12V mutation, the RAS peptide having a length of about 9 to about 11 amino acid residues. The TCRs may have antigenic specificity for a mutated RAS peptide comprising contiguous amino acid residues of mutated RAS protein which include the G12V mutation. In an aspect of the invention, the TCRs may have antigenic specificity for a RAS peptide with the G12V mutation, the mutated RAS peptide having a length of about 9 amino acid residues, about 10 amino acid residues, or about 11 amino acid residues. An example of a specific peptide with the G12V mutation, which may be recognized by the inventive G12V TCRs, is the 10-mer peptide VVVGAVGVGK (SEQ ID NO: 33). In an aspect of the invention, the TCR does not have antigenic specificity for the wild-type human RAS amino acid sequence of VVVGAGGVGK (SEQ ID NO: 34).

In an aspect of the invention, the inventive TCRs are able to recognize mutated RAS presented by an HLA-A3 molecule. In this regard, the TCRs may elicit an immune response upon binding to mutated RAS presented by an HLA-A3 molecule. The inventive TCRs may bind to the HLA-A3 molecule in addition to mutated RAS. The HLA-A3 molecule is a heterodimer of an α chain and β2 microglobulin. The HLA-A3 α chain may be encoded by an HLA-A3 gene. β2 microglobulin binds non-covalently to the alpha1, alpha2 and alpha3 domains of the alpha chain to build the HLA-A3 complex. The HLA-A3 molecule may be any HLA-A3 molecule. Examples of HLA-A3 molecules may include, but are not limited to, HLA-A*3:01, HLA-A*3:02, or HLA-A*3:05.

The TCRs of the invention may provide any one or more of a variety of advantages, including when expressed by cells used for adoptive cell transfer. G12V RAS is expressed by cancer cells and is not expressed by normal, noncancerous cells. Without being bound to a particular theory or mechanism, it is believed that the inventive TCRs advantageously target the destruction of cancer cells while minimizing or eliminating the destruction of normal, non-cancerous cells, thereby reducing, for example, by minimizing or eliminating, toxicity. Moreover, because the G12V mutation is likely to occur in the early stages of tumorigenesis, the G12V RAS mutation may be expressed on substantially all of a patient's cancer cells. The inventive TCRs may, advantageously, successfully treat or prevent G12V RAS-positive cancers that do not respond to other types of treatment such as, for example, chemotherapy, surgery, or radiation. Additionally, the inventive TCRs may provide highly avid recognition of G12V RAS, which may provide the ability to recognize unmanipulated tumor cells (e.g., tumor cells that have not been treated with interferon (IFN)-γ, transfected with a vector encoding one or both of G12V RAS and HLA-A3, pulsed with a G12V RAS peptide, or a combination thereof). The RAS^{G12} mutations are among the most common hotspot mutations found in many cancer types. The KRAS G12V mutation is present in nearly 3.5% of all cancer patients, with pancreatic, lung and colorectal cancer having the greatest prevalence. For example, the KRAS G12V mutation is expressed in about 27% and about 9% of patients with pancreatic and colorectal cancers, respectively. About 30% of patients with KRAS mutations (about 21% of all pancreatic tumor patients) have the KRAS G12V mutation. Moreover, RAS family members share the G12 hotspot mutation in different cancer types (e.g. NRAS in melanoma). Additionally, the inventive TCRs may provide highly avid recognition of mutated RAS, which may provide the ability to recognize unmanipulated tumor cells (e.g., tumor cells that have not been treated with interferon (IFN)-γ, transfected with a vector encoding one or both of mutated RAS and HLA-A3, pulsed with a RAS peptide with the G12V mutation, or a combination thereof). Moreover, the HLA-A3 allele is expressed by about 6% to about 11% of the total U.S. population, including by about 20% to about 30% of the Caucasian population in the United States. Accordingly, the inventive TCRs may increase the number of immunotherapy-eligible cancer patients to include those patients that express HLA-A3 who may not be eligible for immunotherapy using TCRs that recognize mutated RAS presented by other MHC molecules.

The phrase “antigenic specificity.” as used herein, means that the TCR can specifically bind to and immunologically recognize G12V RAS with high avidity. For example, a TCR may be considered to have “antigenic specificity” for G12V RAS if about 1 ×10⁴ to about 1×10⁵ T cells expressing the TCR secrete at least about 200 pg/mL or more (e.g., 200 pg/mL or more, 300 pg/mL or more, 400 pg/mL or more, 500 pg/mL or more, 600 pg/mL or more, 700 pg/mL or more, 1000 pg/mL or more, 5,000 pg/mL or more, 7,000 pg/mL or more, 10,000 pg/mL or more, 20,000 pg/mL or more, or a range defined by any two of the foregoing values) of IFN-γ upon co-culture with antigen-negative, HLA-A3 molecule positive target cells pulsed with a low concentration of G12V RAS peptide (e.g., about 1000 ng/ml or less, 100 ng/ml or less, 10 ng/ml or less, or 1 ng/mL or less, or a range defined by any two of the foregoing values) while co-culture with the antigen-negative, HLA-A3 molecule positive target cells pulsed with a similar concentration of the unmutated wild type peptide results in IFN-gamma secretion of less than half of that amount. In some aspects, the differential is large with IFN-gamma secretion upon co-culture with the target cell pulsed with the wild type peptide being negligible (less than 100 pg/mL) while the IFN-gamma secretion upon co-culture with the target cell pulsed with the mutated G12V peptide being 10 fold higher (or greater). IFN-γ secretion may be measured by methods known in the art such as, for example, enzyme-linked immunosorbent assay (ELISA).

Alternatively or additionally, a TCR may be considered to have “antigenic specificity” for G12V RAS if T cells expressing the TCR secrete at least twice (e.g., ten times) as much IFN-γ upon co-culture with antigen-negative, HLA-A3 molecule positive target cells into which a nucleotide sequence encoding G12V RAS has been introduced such that the target cell expresses G12V RAS as compared to the amount of IFN-γ secretion induced by co-culture with a negative control The negative control may be, for example, (i) T cells expressing the TCR, co-cultured with (a) the unmodified antigen-negative, HLA-A3 molecule positive target cells or (b) the antigen-negative, HLA-A3 molecule positive target cells into which a nucleotide sequence encoding the corresponding sequence of wild type RAS has been introduced. IFN-γ secretion may be measured by methods known in the art such as, for example, enzyme-linked immunosorbent assay (ELISA).

Alternatively or additionally, a TCR may be considered to have “antigenic specificity” for G12V RAS if at least twice (e.g., five times) as many of the numbers of T cells expressing the TCR secrete IFN-γ upon co-culture with (a) antigen-negative, HLA-A3 molecule positive target cells pulsed with a low concentration of G12V RAS peptide or (b) antigen-negative, HLA-A3 molecule positive target cells into which a nucleotide sequence encoding G12V RAS has been introduced such that the target cell expresses G12V RAS as compared to the numbers of negative control T cells that secrete IFN-γ. The HLA-A3 molecule, concentration of peptide, and the negative control may be as described herein with respect to other aspects of the invention. The numbers of cells secreting IFN-γ may be measured by methods known in the art such as, for example, ELISPOT.

Alternatively or additionally, a TCR may be considered to have “antigenic specificity” for G12V RAS if T cells expressing the TCR upregulate expression of one or more T-cell activation markers as measured by, for example, flow cytometry after stimulation with target cells expressing G12V RAS. Examples of T-cell activation markers include 4-1BB, OX40, CD107a, CD69, and cytokines that are upregulated upon antigen stimulation (e.g., tumor necrosis factor (TNF), interleukin (IL)-2, etc.).

An aspect of the invention provides a TCR comprising two polypeptides (i.e., polypeptide chains), such as an alpha (α) chain of a TCR, a beta (β) chain of a TCR, a gamma (γ) chain of a TCR, a delta (δ) chain of a TCR, or a combination thereof. The polypeptides of the inventive TCR can comprise any amino acid sequence, provided that the TCR has antigenic specificity for G12V RAS. In some aspects, the TCR is non-naturally occurring.

In an aspect of the invention, the TCR comprises two polypeptide chains, each of which comprises a variable region comprising a complementarity determining region (CDR)1, a CDR2, and a CDR3 of a TCR. In an aspect of the invention, the TCR comprises a first polypeptide chain comprising a CDR1 comprising the amino acid sequence of SEQ ID NO: 1 (CDR1 of α chain), a CDR2 comprising the amino acid sequence of SEQ ID NO: 2 (CDR2 of α chain), and a CDR3 comprising the amino acid sequence of SEQ ID NO: 3 (CDR3 of α chain), and a second polypeptide chain comprising a CDR1 comprising the amino acid sequence of SEQ ID NO: 4 (CDR1 of β chain), a CDR2 comprising the amino acid sequence of SEQ ID NO: 5 (CDR2 of β chain), and a CDR3 comprising the amino acid sequence of SEQ ID NO: 6 (CDR3 of β chain). In this regard, the inventive TCR can comprise any one or more of the amino acid sequences selected from the group consisting of SEQ ID NOs: 1-6. In an aspect of the invention, the TCR comprises the amino acid sequences of: (a) all of SEQ ID NOs: 1-3, (b) all of SEQ ID NOs: 4-6, or (c) all of SEQ ID NOs: 1-6. In an especially preferred aspect, the TCR comprises the amino acid sequences of all of SEQ ID NOs: 1-6.

In an aspect of the invention, the TCR comprises an amino acid sequence of a variable region of a TCR comprising the CDRs set forth above. In this regard, the TCR can comprise the amino acid sequence of: (i) SEQ ID NO: 7 (predicted sequence of variable region of α chain without N-terminal signal peptide); (ii) SEQ ID NO: 8 (predicted sequence of variable region of β chain without N-terminal signal peptide); (iii) SEQ ID NO: 9 (variable region of a chain with N-terminal signal peptide); (iv) SEQ ID NO: 10 (variable region of β chain with N-terminal signal peptide); (v) both of SEQ ID NOs: 7 and 8; or (vi) both of SEQ ID NOs: 9 and 10.

The inventive TCRs may further comprise an a chain constant region and a β chain constant region. The constant region may be derived from any suitable species such as, e.g., human or mouse. In an aspect of the invention, the TCRs further comprise murine α and β chain constant regions or human α and β chain constant regions. As used herein, the term “murine” or “human,” when referring to a TCR or any component of a TCR described herein (e.g., CDR, variable region, constant region, α chain, and/or β chain), means a TCR (or component thereof) which is derived from a mouse or a human, respectively, i.e., a TCR (or component thereof) that originated from or was, at one time, expressed by a mouse T cell or a human T cell, respectively.

An aspect of the invention provides a murine TCR comprising a murine variable region and a murine constant region, wherein the TCR has antigenic specificity for a mutated human RAS amino acid sequence presented by an HLA-A3 molecule. The murine constant region may provide any one or more advantages. For example, the murine constant region may diminish mispairing of the inventive TCR with the endogenous TCRs of the host cell into which the inventive TCR is introduced when the host cell is not a murine host cell, e.g., a human host cell. Alternatively or additionally, the murine constant region may increase expression of the inventive TCR as compared to the same TCR with a human constant region. The TCR may comprise the amino acid sequence of SEQ ID NO: 21 (wild-type (WT) murine α chain constant region), SEQ ID NO: 22 (WT murine β chain constant region), or both SEQ ID NOs: 21 and 22. Preferably, the inventive TCR comprises the amino acid sequences of both of SEQ ID NOs: 21 and 22. The TCR may comprise any of the murine constant regions described herein in combination with any of the CDR regions as described herein with respect to other aspects of the invention. In this regard, the TCR may comprise the amino acid sequences of: (a) all of SEQ ID NOs: 1-3 and 21; (b) all of SEQ ID NOs: 4-6 and 22; or (c) all of SEQ ID NOs: 1-6 and 21-22. In another aspect of the invention, the TCR may comprise any of the murine constant regions described herein in combination with any of the variable regions described herein with respect to other aspects of the invention. In this regard, the TCR may comprise the amino acid sequences of: (i) both of SEQ ID NOs: 7 and 21; (ii) both of SEQ ID NOs: 8 and 22; (iii) both of SEQ ID NOs: 9 and 21; (iv) both of SEQ ID NOs: 10 and 22; (v) all of SEQ ID NOs: 7-8 and 21-22; or (vi) all of SEQ ID NOs: 9-10 and 21-22.

In an aspect of the invention, the TCR comprises a substituted constant region. In this regard, the TCR may comprise the amino acid sequence of any of the TCRs described herein with one, two, three, or four amino acid substitution(s) in the constant region of one or both of the α and β chain. Preferably, the TCR comprises a murine constant region with one, two, three, or four amino acid substitution(s) in the murine constant region of one or both of the α and β chains. In an especially preferred aspect, the TCR comprises a murine constant region with one, two, three, or four amino acid substitution(s) in the murine constant region of the α chain and one amino acid substitution in the murine constant region of the β chain. In some aspects, the TCRs comprising the substituted constant region may provide one or more of increased recognition of G12V RAS⁺ targets, increased expression by a host cell, diminished mispairing with endogenous TCRs, and increased anti-tumor activity as compared to the parent TCR comprising an unsubstituted (wild-type) constant region. In general, the substituted amino acid sequences of the murine constant regions of the TCR α and β chains, SEQ ID NOs: 19 and 20, respectively, correspond with all or portions of the unsubstituted murine constant region amino acid sequences SEQ ID NOs: 21 and 22, respectively, with SEQ ID NO: 19 having one, two, three, or four amino acid substitution(s) when compared to SEQ ID NO: 21 and SEQ ID NO: 20 having one amino acid substitution when compared to SEQ ID NO: 22. In this regard, an aspect of the invention provides a TCR comprising the amino acid sequences of (a) SEQ ID NO: 19 (constant region of α chain), wherein (i) X at position 48 is Thr or Cys; (ii) X at position 112 is Ser, Ala, Val, Leu, Ile, Pro, Phe, Met, or Trp; (iii) X at position 114 is Met, Ala, Val, Leu, Ile, Pro, Phe, or Trp; and (iv) X at position 115 is Gly, Ala, Val, Leu, Ile, Pro, Phe, Met, or Trp; (b) SEQ ID NO: 20 (constant region of β chain), wherein X at position 57 is Ser or Cys; or (c) both of SEQ ID NOs: 19 and 20. In an aspect of the invention, the TCR comprising SEQ ID NO: 19 does not comprise SEQ ID NO: 21 (unsubstituted murine constant region of α chain). In an aspect of the invention, the TCR comprising SEQ ID NO: 20 does not comprise SEQ ID NO: 22 (unsubstituted murine constant region of β chain).

In an aspect of the invention, the TCR comprises an α chain comprising a variable region and a constant region and a β chain comprising a variable region and a constant region. In this regard, the TCR may comprise: (a) an α chain comprising the amino acid sequence of SEQ ID NO: 23 (α chain with N-terminal signal peptide), wherein: (i) X at position 181 of SEQ ID NO: 23 is Thr or Cys; (ii) X at position 245 of SEQ ID NO: 23 is Ser, Ala, Val, Leu, Ile, Pro, Phe, Met, or Trp; (iii) X at position 247 of SEQ ID NO: 23 is Met, Ala, Val, Leu, Ile, Pro, Phe, or Trp; and (iv) X at position 248 of SEQ ID NO: 23 is Gly, Ala, Val, Leu, Ile, Pro, Phe, Met, or Trp; (b) a β chain comprising the amino acid sequence of SEQ ID NO: 24 (β chain with N-terminal signal peptide), wherein X at position 188 of SEQ ID NO: 24 is Ser or Cys; (c) both of SEQ ID NOs: 23 and 24; (d) an α chain comprising the amino acid sequence of SEQ ID NO: 25 (predicted sequence of α chain without N-terminal signal peptide), wherein: (i) X at position 161 of SEQ ID NO: 25 is Thr or Cys; (ii) X at position 225 of SEQ ID NO: 25 is Ser, Ala, Val, Leu, Ile, Pro, Phe, Met, or Trp; (iii) X at position 227 of SEQ ID NO: 25 is Met, Ala, Val, Leu, Ile, Pro, Phe, or Trp; and (iv) X at position 228 of SEQ ID NO: 25 is Gly, Ala, Val, Leu, Ile, Pro, Phe, Met, or Trp; (e) a β chain comprising the amino acid sequence of SEQ ID NO: 26 (predicted sequence of β chain without N-terminal signal peptide), wherein X at position 170 of SEQ ID NO: 26 is Ser or Cys; (f) both of SEQ ID NOs: 25 and 26; (g) SEQ ID NO: 27 (α chain with N-terminal signal peptide and unsubstituted murine constant region); (h) SEQ ID NO: 28 (β chain with N-terminal signal peptide and unsubstituted murine constant region); (i) SEQ ID NO: 29 (predicted sequence of α chain with unsubstituted murine constant region and without N-terminal signal peptide); (j) SEQ ID NO: 30 (predicted sequence of β chain with unsubstituted murine constant region and without N-terminal signal peptide); (k) both of SEQ ID NOs: 27 and 28; or (l) both of SEQ ID NOs: 29 and 30.

In an aspect of the invention, the substituted constant region includes cysteine substitutions in the constant region of one or both of the α and β chains to provide a cysteine-substituted TCR. Opposing cysteines in the α and the β chains provide a disulfide bond that links the constant regions of the α and the β chains of the substituted TCR to one another and which is not present in a TCR comprising the unsubstituted murine constant regions. In this regard, the TCR may be a cysteine-substituted TCR in which one or both of the native Thr at position 48 (Thr48) of SEQ ID NO: 21 and the native Ser at position 57 (Ser57) of SEQ ID NO: 22 may be substituted with Cys. Preferably, both of the native Thr48 of SEQ ID NO: 21 and the native Ser57 of SEQ ID NO: 22 are substituted with Cys. Examples of cysteine-substituted TCR constant regions sequences are set forth in Table 2. In an aspect of the invention, the cysteine-substituted TCR comprises (i) SEQ ID NO: 19, (ii) SEQ ID NO: 20, or (iii) both of SEQ ID NOs: 19 and 20, wherein both of SEQ ID NOs: 19 and 20 are as defined in Table 2. The cysteine-substituted TCRs of the invention may include the substituted constant region in addition to any of the CDRs or variable regions described herein.

In an aspect of the invention, the cysteine-substituted TCR comprises a full length a chain and a full-length β chain. Examples of cysteine-substituted TCR α chain and β chain sequences are set forth in Table 2. In an aspect of the invention, the TCR comprises: (i) SEQ ID NO: 23, (ii) SEQ ID NO: 24, (iii) SEQ ID NO: 25, (iv) SEQ ID NO: 26, (v) both of SEQ ID NOs: 23 and 24, or (vi) both of SEQ ID NO: 25 and 26, wherein all of SEQ ID NOs: 23-26 are as defined in Table 2.

TABLE 2

| SEQ ID NO: | Definitions of “X” |
|---|---|
| SEQ ID NO: 19 | X at position 48 is Cys, |
| (constant region α chain) | X at position 112 is Ser, |
| | X at position 114 is Met, and |
| | X at position 115 is Gly. |
| SEQ ID NO: 20 | X at position 57 is Cys |
| (constant region β chain) | |
| SEQ ID NO: 23 | X at position 181 is Cys, |
| (α chain with N-terminal | X at position 245 is Ser, |
| signal peptide) | X at position 247 is Met, and |
| | X at position 248 is Gly. |
| SEQ ID NO: 24 | X at position 188 is Cys |
| (β chain with N-terminal | |
| signal peptide) | |
| SEQ ID NO: 25 | X at position 161 is Cys, |
| (α chain predicted sequence | X at position 225 is Ser, |
| without N-terminal signal | X at position 227 is Met, and |
| peptide) | X at position 228 is Gly. |
| SEQ ID NO: 26 | X at position 170 is Cys |
| (β chain predicted sequence | |
| without N-terminal signal | |
| peptide) | |

In an aspect of the invention, the substituted amino acid sequence includes substitutions of one, two, or three amino acids in the transmembrane (TM) domain of the constant region of the α chain with a hydrophobic amino acid to provide a hydrophobic amino acid-substituted TCR (also referred to herein as an “LVL-modified TCR”). The hydrophobic amino acid substitution(s) in the TM domain of the TCR may increase the hydrophobicity of the TM domain of the TCR as compared to a TCR that lacks the hydrophobic amino acid substitution(s) in the TM domain. In this regard, the TCR is an LVL-modified TCR in which one, two, or three of the native Ser112, Met114, and Gly115 of SEQ ID NO: 21 may, independently, be substituted with Ala, Val, Leu, Ile, Pro, Phe, Met, or Trp; preferably with Leu, Ile, or Val. Preferably, all three of the native Ser112, Met114, and Gly115 of SEQ ID NO: 21 may, independently, be substituted with Ala, Val, Leu, Ile, Pro, Phe, Met, or Trp; preferably with Leu, Ile, or Val. In an aspect of the invention, the LVL-modified TCR comprises (i) SEQ ID NO: 19, (ii) SEQ ID NO: 20, or (iii) both of SEQ ID NOs: 19 and 20, wherein both of SEQ ID NOs: 19 and 20 are as defined in Table 3. The LVL-modified TCRs of the invention may include the substituted constant region in addition to any of the CDRs or variable regions described herein.

In an aspect of the invention, the LVL-modified TCR comprises a full length α chain and a full-length β chain. Examples of LVL-modified TCR α chain and β chain sequences are set forth in Table 3. In an aspect of the invention, the TCR comprises: (i) SEQ ID NO: 23, (ii) SEQ ID NO: 24, (iii) SEQ ID NO: 25, (iv) SEQ ID NO: 26, (v) both of SEQ ID NOs: 23 and 24, or (vi) both of SEQ ID NOs: 25 and 26, wherein all of SEQ ID NOs: 23-26 are as defined in Table 3.

TABLE 3

| SEQ ID NO: | Definitions of “X” |
|---|---|
| SEQ ID NO: 19 | X at position 48 is Thr; |
| (constant region α | X at position 112 is Ser, Ala, Val, Leu, Ile, Pro, Phe, Met, or Trp; |
| chain) | preferably wherein X at position 112 is Leu, Ile, or Val; |
| | especially preferably wherein X at position 112 is Leu; |
| | X at position 114 is Met, Ala, Val, Leu, Ile, Pro, Phe, or Trp; |
| | preferably wherein X at position 114 is Leu, Ile, or Val; |
| | especially preferably wherein X at position 114 is Ile; and |
| | X at position 115 is Gly, Ala, Val, Leu, Ile, Pro, Phe, Met, or Trp; |
| | preferably wherein X at position 115 is Leu, Ile, or Val; |
| | especially preferably wherein X at position 115 is Val; |
| | wherein SEQ ID NO: 19 does not comprise SEQ ID NO: 21 (unsubstituted |
| | α chain constant region) |
| SEQ ID NO: 20 | X at position 57 is Ser |
| (constant region β | |
| chain) | |
| SEQ ID NO: 23 (α | X at position 181 is Thr; |
| chain) (with N- | X at position 245 is Ser, Ala, Val, Leu, Ile, Pro, Phe, Met, or Trp; |
| terminal signal | preferably wherein X at position 245 is Leu, Ile, or Val; |
| peptide) | especially preferably wherein X at position 245 is Leu; |
| | X at position 247 is Met, Ala, Val, Leu, Ile, Pro, Phe, or Trp; |
| | preferably wherein X at position 247 is Leu, Ile, or Val; |
| | especially preferably wherein X at position 247 is Ile; and |
| | X at position 248 is Gly, Ala, Val, Leu, Ile, Pro, Phe, Met, or Trp; |
| | preferably wherein X at position 248 is Leu, Ile, or Val; |
| | especially preferably wherein X at position 248 is Val, |
| | wherein SEQ ID NO: 23 does not comprise SEQ ID NO: 21 (unsubstituted |
| | α chain constant region) |
| SEQ ID NO: 24 (β | X at position 188 is Ser |
| chain) (with N- | |
| terminal signal | |
| peptide) | |
| SEQ ID NO: 25 (α | X at position 161 is Thr; |
| chain) (predicted | X at position 225 is Ser, Ala, Val, Leu, Ile, Pro, Phe, Met, or Trp; |
| sequence without N- | preferably wherein X at position 225 is Leu, Ile, or Val; |
| terminal signal | especially preferably wherein X at position 225 is Leu; |
| peptide) | X at position 227 is Met, Ala, Val, Leu, Ile, Pro, Phe, or Trp; |
| | preferably wherein X at position 227 is Leu, Ile, or Val; |
| | especially preferably wherein X at position 227 is Ile; and |
| | X at position 228 is Gly, Ala, Val, Leu, Ile, Pro, Phe, Met, or Trp; |
| | preferably wherein X at position 228 is Leu, Ile, or Val; |
| | especially preferably wherein X at position 228 is Val, |
| | wherein SEQ ID NO: 25 does not comprise SEQ ID NO: 21 (unsubstituted |
| | α chain constant region) |
| SEQ ID NO: 26 (β | X at position 170 is Ser |
| chain) (predicted | |
| sequence without N- | |
| terminal signal | |
| peptide) | |

In an aspect of the invention, the substituted amino acid sequence includes the cysteine substitutions in the constant region of one or both of the α and β chains in combination with the substitution(s) of one, two, or three amino acids in the transmembrane (TM) domain of the constant region of the α chain with a hydrophobic amino acid (also referred to herein as “cysteine-substituted, LVL-modified TCR”). In this regard, the TCR is a cysteine-substituted, LVL-modified TCR in which the native Thr48 of SEQ ID NO: 21 is substituted with Cys; one, two, or three of the native Ser112, Met114, and Gly115 of SEQ ID NO: 21 are, independently, substituted with Ala, Val, Leu, Ile, Pro, Phe, Met, or Trp; preferably with Leu, Ile, or Val; and the native Ser57 of SEQ ID NO: 22 is substituted with Cys. Preferably, all three of the native Ser112, Met114, and Gly115 of SEQ ID NO: 21 may, independently, be substituted with Ala, Val, Leu, Ile, Pro, Phe, Met, or Trp; preferably with Leu, Ile, or Val. In an aspect of the invention, the cysteine-substituted, LVL-modified TCR comprises (i) SEQ ID NO: 19, (ii) SEQ ID NO: 20, or (iii) both of SEQ ID NOs: 19 and 20, wherein both of SEQ ID NOs: 19 and 20 are as defined in Table 4. The cysteine-substituted, LVL-modified TCRs of the invention may include the substituted constant region in addition to any of the CDRs or variable regions described herein.

In an aspect, the cysteine-substituted, LVL-modified TCR comprises a full-length α chain and a full-length β chain. Examples of cysteine-substituted, LVL-modified TCR α chain and β chain sequences are set forth in Table 4. In an aspect of the invention, the TCR comprises: (1) SEQ ID NO: 23, (2) SEQ ID NO: 24, (3) SEQ ID NO: 25, (4) SEQ ID NO: 26, (5) both of SEQ ID NOs: 23 and 24, or (6) both of SEQ ID NOs: 25 and 26, wherein all of SEQ ID NOs: 23-26 are as defined in Table 4.

TABLE 4

| SEQ ID NO: | Definitions of “X” |
|---|---|
| SEQ ID NO: 19 | X at position 48 is Cys; |
| (constant region α | X at position 112 is Ser, Ala, Val, Leu, Ile, Pro, Phe, Met, or Trp; |
| chain) | preferably wherein X at position 112 is Leu, Ile, or Val; |
| | especially preferably wherein X at position 112 is Leu; |
| | X at position 114 is Met, Ala, Val, Leu, Ile, Pro, Phe, or Trp; |
| | preferably wherein X at position 114 is Leu, Ile, or Val; |
| | especially preferably wherein X at position 114 is Ile; and |
| | X at position 115 is Gly, Ala, Val, Leu, Ile, Pro, Phe, Met, or Trp; |
| | preferably wherein X at position 115 is Leu, Ile, or Val; and |
| | especially preferably wherein X at position 115 is Val, |
| | wherein SEQ ID NO: 19 does not simultaneously comprise all of Ser at |
| | position 112, Met at position 114, and Gly at position 115. |
| SEQ ID NO: 20 | X at position 57 is Cys |
| (constant region β | |
| chain) | |
| SEQ ID NO: 23 (α | X at position 181 is Cys; |
| chain) (with N-terminal | X at position 245 is Ser, Ala, Val, Leu, Ile, Pro, Phe, Met, or Trp; |
| signal peptide) | preferably wherein X at position 245 is Leu, Ile, or Val; |
| | especially preferably wherein X at position 245 is Leu; |
| | X at position 247 is Met, Ala, Val, Leu, Ile, Pro, Phe, or Trp; |
| | preferably wherein X at position 247 is Leu, Ile, or Val; |
| | especially preferably wherein X at position 247 is Ile; and |
| | X at position 248 is Gly, Ala, Val, Leu, Ile, Pro, Phe, Met, or Trp; |
| | preferably wherein X at position 248 is Leu, Ile, or Val; and |
| | especially preferably wherein X at position 248 is Val, |
| | wherein SEQ ID NO: 23 does not simultaneously comprise all of Ser at |
| | position 245, Met at position 247, and Gly at position 248. |
| SEQ ID NO: 24 (β | X at position 188 is Cys |
| chain) (with N-terminal | |
| signal peptide) | |
| SEQ ID NO: 25 (α | X at position 161 is Cys; |
| chain) (predicted | X at position 225 is Ser, Ala, Val, Leu, Ile, Pro, Phe, Met, or Trp; |
| sequence without N- | preferably wherein X at position 225 is Leu, Ile, or Val; |
| terminal signal peptide) | especially preferably wherein X at position 225 is Leu; |
| | X at position 227 is Met, Ala, Val, Leu, Ile, Pro, Phe, or Trp; |
| | preferably wherein X at position 227 is Leu, Ile, or Val; |
| | especially preferably wherein X at position 227 is Ile; and |
| | X at position 228 is Gly, Ala, Val, Leu, Ile, Pro, Phe, Met, or Trp; |
| | preferably wherein X at position 228 is Leu, Ile, or Val; and |
| | especially preferably wherein X at position 228 is Val, |
| | wherein SEQ ID NO: 25 does not simultaneously comprise all of Ser at |
| | position 225, Met at position 227, and Gly at position 228. |
| SEQ ID NO: 26 (β | X at position 170 is Cys |
| chain) (predicted | |
| sequence without N- | |
| terminal signal peptide) | |

Also provided by the invention is a polypeptide comprising a functional portion of any of the TCRs described herein. The term “polypeptide,” as used herein, includes oligopeptides and refers to a single chain of amino acids connected by one or more peptide bonds.

With respect to the inventive polypeptides, the functional portion can be any portion comprising contiguous amino acids of the TCR of which it is a part, provided that the functional portion specifically binds to G12V RAS. The term “functional portion,” when used in reference to a TCR, refers to any part or fragment of the TCR of the invention, which part or fragment retains the biological activity of the TCR of which it is a part (the parent TCR). Functional portions encompass, for example, those parts of a TCR that retain the ability to specifically bind to G12V RAS (e.g., presented by an HLA-A3 molecule), or detect, treat, or prevent cancer, to a similar extent, the same extent, or to a higher extent, as the parent TCR. In reference to the parent TCR, the functional portion can comprise, for instance, about 10%, about 25%, about 30%, about 50%, about 70%, about 80%, about 90%, about 95%, or more, of the parent TCR.

The functional portion can comprise additional amino acids at the amino or carboxy terminus of the portion, or at both termini, which additional amino acids are not found in the amino acid sequence of the parent TCR. Desirably, the additional amino acids do not interfere with the biological function of the functional portion, e.g., specifically binding to G12V RAS; and/or having the ability to detect cancer, treat or prevent cancer, etc. More desirably, the additional amino acids enhance the biological activity, as compared to the biological activity of the parent TCR.

The polypeptide can comprise a functional portion of either or both of the α and β chains of the TCRs of the invention, such as a functional portion comprising one or more of the CDR1, CDR2, and CDR3 of the variable region(s) of the a chain and/or β chain of a TCR of the invention. In an aspect of the invention, the polypeptide can comprise the amino acid sequence of SEQ ID NO: 1 (CDR1 of α chain), SEQ ID NO: 2 (CDR2 of α chain), SEQ ID NO: 3 (CDR3 of α chain), SEQ ID NO: 4 (CDR1 of β chain), SEQ ID NO: 5 (CDR2 of β chain), SEQ ID NO: 6 (CDR3 of β chain), or a combination thereof. In this regard, the inventive polypeptide can comprise any one or more of the amino acid sequences selected from the group consisting of SEQ ID NOs: 1-6. In an aspect of the invention, the TCR comprises the amino acid sequences of: (a) all of SEQ ID NOs: 1-3, (b) all of SEQ ID NOs: 4-6, or (c) all of SEQ ID NOs: 1-6. In a preferred aspect, the polypeptide comprises the amino acid sequences of all of SEQ ID NOs: 1-6.

In an aspect of the invention, the inventive polypeptide can comprise, for instance, the variable region of the inventive TCR comprising a combination of the CDR regions set forth above. In this regard, the polypeptide can comprise the amino acid sequence of (i) SEQ ID NO: 7 (predicted sequence of variable region of α chain without N-terminal signal peptide); (ii) SEQ ID NO: 8 (predicted sequence of variable region of β chain without N-terminal signal peptide); (iii) SEQ ID NO: 9 (variable region of α chain with N-terminal signal peptide); (iv) SEQ ID NO: 10 (variable region of β chain with N-terminal signal peptide); (v) both of SEQ ID NOs: 7 and 8; or (vi) both of SEQ ID NOs: 9 and 10. Preferably, the polypeptide comprises the amino acid sequences of (i) both of SEQ ID NOs: 7 and 8 or (ii) both of SEQ ID NOs: 9 and 10.

In an aspect of the invention, the inventive polypeptide can further comprise the constant region of the inventive TCR set forth above. In this regard, the polypeptide can further comprise the amino acid sequence of (i) SEQ ID NO: 21 (WT murine constant region of α chain), (ii) SEQ ID NO: 22 (WT murine constant region of β chain), (iii) SEQ ID NO: 19 (substituted murine constant region of α chain), (iv) SEQ ID NO: 20 (substituted murine constant region of β chain), (v) both SEQ ID NOs: 19 and 20, or (vi) both SEQ ID NOs: 21 and 22. Preferably, the polypeptide further comprises the amino acid sequences of (i) both of SEQ ID NOs: 19 and 20 or (ii) both of SEQ ID NO: 21 and 22 in combination with any of the CDR regions or variable regions described herein with respect to other aspects of the invention. In an aspect of the invention, one or both of SEQ ID NOs: 19 and 20 of the polypeptide are as defined in any one of Tables 2-4.

In an aspect of the invention, the inventive polypeptide can comprise the entire length of an α or β chain of the TCR described herein. In this regard, the inventive polypeptide can comprise the amino acid sequence of SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30. Alternatively, the polypeptide of the invention can comprise both chains of the TCRs described herein. For example, the polypeptide may comprise both of SEQ ID NOs: 23 and 24, both of SEQ ID NOs: 25 and 26, both of SEQ ID NOs: 27 and 28, or both of SEQ ID NOs: 29 and 30.

For example, the polypeptide of the invention can comprise (a) the amino acid sequence of SEQ ID NO: 23 (α chain with N-terminal signal peptide), wherein: (i) X at position 181 of SEQ ID NO: 23 is Thr or Cys; (ii) X at position 245 of SEQ ID NO: 23 is Ser, Ala, Val, Leu, Ile, Pro, Phe, Met, or Trp; (iii) X at position 247 of SEQ ID NO: 23 is Met, Ala, Val, Leu, Ile, Pro, Phe, or Trp; and (iv) X at position 248 of SEQ ID NO: 23 is Gly, Ala, Val, Leu, Ile, Pro, Phe, Met, or Trp; (b) the amino acid sequence of SEQ ID NO: 24 (β chain with N-terminal signal peptide), wherein X at position 188 of SEQ ID NO: 24 is Ser or Cys; (c) both of SEQ ID NOs: 23 and 24; (d) the amino acid sequence of SEQ ID NO: 25 (predicted sequence of aα chain without N-terminal signal peptide), wherein: (i) X at position 161 of SEQ ID NO: 25 is Thr or Cys; (ii) X at position 225 of SEQ ID NO: 25 is Ser, Ala, Val, Leu, Ile, Pro, Phe, Met, or Trp; (iii) X at position 227 of SEQ ID NO: 25 is Met, Ala, Val, Leu, Ile, Pro, Phe, or Trp; and (iv) X at position 228 of SEQ ID NO: 25 is Gly, Ala, Val, Leu, Ile, Pro, Phe, Met, or Trp; (e) the amino acid sequence of SEQ ID NO: 26 (predicted sequence of β chain without N-terminal signal peptide), wherein X at position 170 of SEQ ID NO: 26 is Ser or Cys; (f) both of SEQ ID NOs: 25 and 26; (g) SEQ ID NO: 27; (h) SEQ ID NO: 28; (i) SEQ ID NO: 29; (j) SEQ ID NO: 30; (k) both of SEQ ID NOs: 27 and 28; or (l) both of SEQ ID NOs: 29 and 30. In an aspect of the invention, any one or more of SEQ ID NOs: 23-26 of the polypeptide are as defined in any one of Tables 2-4.

The invention further provides a protein comprising at least one of the polypeptides described herein. By “protein” is meant a molecule comprising one or more polypeptide chains.

In an aspect, the protein of the invention can comprise (a) a first polypeptide chain comprising the amino acid sequences of SEQ ID NOs: 1-3; (b) a second polypeptide chain comprising the amino acid sequences of SEQ ID NOs: 4-6; or (c) a first polypeptide chain comprising the amino acid sequences of SEQ ID NOs: 1-3 and a second polypeptide chain comprising the amino acid sequences of SEQ ID NOs: 4-6.

In another aspect of the invention, (a) the first polypeptide chain comprises the amino acid sequence of SEQ ID NO: 7; (b) the second polypeptide chain comprises the amino acid sequence of SEQ ID NO: 8; (c) the first polypeptide chain comprises the amino acid sequence of SEQ ID NO: 9; (d) the second polypeptide chain comprises the amino acid sequence of SEQ ID NO: 10; (e) the first polypeptide chain comprises the amino acid sequence of SEQ ID NO: 7 and the second polypeptide chain comprises the amino acid sequence of SEQ ID NO: 8; or (f) the first polypeptide chain comprises the amino acid sequence of SEQ ID NO: 9 and the second polypeptide chain comprises the amino acid sequence of SEQ ID NO: 10.

The inventive protein may further comprise any of the constant regions described herein with respect to other aspects of the invention. In this regard, in an aspect of the invention, (i) the first polypeptide chain may further comprise the amino acid sequence of SEQ ID NO: 19; (ii) the second polypeptide chain may further comprise the amino acid sequence of SEQ ID NO: 20; (iii) the first polypeptide chain may further comprise the amino acid sequence of SEQ ID NO: 21; (iv) the second polypeptide chain may further comprise the amino acid sequence of SEQ ID NO: 22; (v) the first polypeptide chain may further comprise the amino acid sequence of SEQ ID NO: 19 and the second polypeptide chain may further comprise the amino acid sequence of SEQ ID NO: 20; or (vi) the first polypeptide chain may further comprise the amino acid sequence of SEQ ID NO: 21 and the second polypeptide chain may further comprise the amino acid sequence of SEQ ID NO: 22. In an aspect of the invention, one or both of SEQ ID NOs: 19 and 20 of the protein are as defined in any one of Tables 2-4.

The inventive protein may comprise a full length α or β chain, as described herein with respect to other aspects of the invention. In this regard, in an aspect of the invention, (a) the first polypeptide chain comprises the amino acid sequence of SEQ ID NO: 23, wherein: (i) X at position 181 of SEQ ID NO: 23 is Thr or Cys; (ii) X at position 245 of SEQ ID NO: 23 is Ser, Ala, Val, Leu, Ile, Pro, Phe, Met, or Trp; (iii) X at position 247 of SEQ ID NO: 23 is Met, Ala, Val, Leu, Ile, Pro, Phe, or Trp; and (iv) X at position 248 of SEQ ID NO: 23 is Gly, Ala, Val, Leu, Ile, Pro, Phe, Met, or Trp; (b) the second polypeptide chain comprises the amino acid sequence of SEQ ID NO: 24, wherein X at position 188 of SEQ ID NO: 24 is Ser or Cys; (c) the first polypeptide chain comprises the amino acid sequence of SEQ ID NO: 23 and the second polypeptide chain comprises the amino acid sequence of SEQ ID NO: 24; (d) the first polypeptide chain comprises the amino acid sequence of SEQ ID NO: 25, wherein: (i) X at position 161 of SEQ ID NO: 25 is Thr or Cys; (ii) X at position 225 of SEQ ID NO: 25 is Ser, Ala, Val, Leu, Ile, Pro, Phe, Met, or Trp; (iii) X at position 227 of SEQ ID NO: 25 is Met, Ala, Val, Leu, Ile, Pro, Phe, or Trp; and (iv) X at position 228 of SEQ ID NO: 25 is Gly, Ala, Val, Leu, Ile, Pro, Phe, Met, or Trp; (e) the second polypeptide chain comprises the amino acid sequence of SEQ ID NO: 26, wherein X at position 170 of SEQ ID NO: 26 is Ser or Cys; (f) the first polypeptide chain comprises the amino acid sequence of SEQ ID NO: 25 and the second polypeptide chain comprises the amino acid sequence of SEQ ID NO: 26; (g) the first polypeptide chain comprises the amino acid sequence of SEQ ID NO: 27; (h) the second polypeptide chain comprises the amino acid sequence of SEQ ID NO: 28; (i) the first polypeptide chain comprises the amino acid sequence of SEQ ID NO: 29; (j) the second polypeptide chain comprises the amino acid sequence of SEQ ID NO: 30; (k) the first polypeptide chain comprises the amino acid sequence of SEQ ID NO: 27 and the second polypeptide chain comprises the amino acid sequence of SEQ ID NO: 28; or (l) the first polypeptide chain comprises the amino acid sequence of SEQ ID NO: 29 and the second polypeptide chain comprises the amino acid sequence of SEQ ID NO: 30. In an aspect of the invention, one or more of SEQ ID NOs: 23-26 are as defined in any one of Tables 2-4.

The protein of the invention can be a TCR. Alternatively, if, for example, the protein comprises a single polypeptide chain comprising the amino acid sequences of both the TCR α and β chains, or if the first and/or second polypeptide chain(s) of the protein further comprise(s) other amino acid sequences, e.g., an amino acid sequence encoding an immunoglobulin or a portion thereof, then the inventive protein can be a fusion protein. In this regard, the invention also provides a fusion protein comprising at least one of the inventive polypeptides described herein along with at least one other polypeptide. The other polypeptide can exist as a separate polypeptide of the fusion protein, or can exist as a polypeptide, which is expressed in frame (in tandem) with one of the inventive polypeptides described herein. The other polypeptide can encode any peptidic or proteinaceous molecule, or a portion thereof, including, but not limited to an immunoglobulin, CD3, CD4, CD8, an MHC molecule, a CD1 molecule, e.g., CD1a, CD1b, CD1c, CD1d, etc.

The fusion protein can comprise one or more copies of the inventive polypeptide and/or one or more copies of the other polypeptide. For instance, the fusion protein can comprise 1, 2, 3, 4, 5, or more, copies of the inventive polypeptide and/or of the other polypeptide. Suitable methods of making fusion proteins are known in the art, and include, for example, recombinant methods.

In some aspects of the invention, the TCRs, polypeptides, and proteins of the invention may be expressed as a single protein comprising a linker peptide linking the α chain and the β chain. In this regard, the TCRs, polypeptides, and proteins of the invention may further comprise a linker peptide. The linker peptide may advantageously facilitate the expression of a recombinant TCR, polypeptide, and/or protein in a host cell. The linker peptide may comprise any suitable amino acid sequence. The linker peptide may be a cleavable linker peptide. For example, the linker peptide may be a furin-SGSG-P2A linker peptide comprising the amino acid sequence of RAKRSGSGATNFSLLKQAGDVEENPGP (SEQ ID NO: 31). Upon expression of the construct including the linker peptide by a host cell, the linker peptide may be cleared, resulting in separated α and β chains. In an aspect of the invention, the TCR, polypeptide, or protein may comprise an amino acid sequence comprising a full-length a chain, a full-length β chain, and a linker peptide positioned between the α and β chains.

The protein of the invention can be a recombinant antibody, or an antigen binding portion thereof, comprising at least one of the inventive polypeptides described herein. As used herein, “recombinant antibody” refers to a recombinant (e.g., genetically engineered) protein comprising at least one of the polypeptides of the invention and a polypeptide chain of an antibody, or an antigen binding portion thereof. The polypeptide of an antibody, or antigen binding portion thereof, can be a heavy chain, a light chain, a variable or constant region of a heavy or light chain, a single chain variable fragment (scFv), or an Fc, Fab, or F(ab)₂′ fragment of an antibody, etc. The polypeptide chain of an antibody, or an antigen binding portion thereof, can exist as a separate polypeptide of the recombinant antibody. Alternatively, the polypeptide chain of an antibody, or an antigen binding portion thereof, can exist as a polypeptide, which is expressed in frame (in tandem) with the polypeptide of the invention. The polypeptide of an antibody, or an antigen binding portion thereof, can be a polypeptide of any antibody or any antibody fragment, including any of the antibodies and antibody fragments described herein.

Included in the scope of the invention are functional variants of the inventive TCRs, polypeptides, or proteins described herein. The term “functional variant,” as used herein, refers to a TCR, polypeptide, or protein having substantial or significant sequence identity or similarity to a parent TCR, polypeptide, or protein, which functional variant retains the biological activity of the TCR, polypeptide, or protein of which it is a variant. Functional variants encompass, for example, those variants of the TCR, polypeptide, or protein described herein (the parent TCR, polypeptide, or protein) that retain the ability to specifically bind to the G12V RAS for which the parent TCR has antigenic specificity or to which the parent polypeptide or protein specifically binds, to a similar extent, the same extent, or to a higher extent, as the parent TCR, polypeptide, or protein. In reference to the parent TCR, polypeptide, or protein, the functional variant can, for instance, be at least about 30%, about 50%, about 75%, about 80%, about 90%, about 95%, about 96%, about 97%, about 98%, about 99% or more identical in amino acid sequence to the parent TCR, polypeptide, or protein, respectively.

The functional variant can, for example, comprise the amino acid sequence of the parent TCR, polypeptide, or protein with at least one conservative amino acid substitution. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

Alternatively or additionally, the functional variants can comprise the amino acid sequence of the parent TCR, polypeptide, or protein with at least one non-conservative amino acid substitution. In this case, it is preferable for the non-conservative amino acid substitution to not interfere with or inhibit the biological activity of the functional variant. Preferably, the non-conservative amino acid substitution enhances the biological activity of the functional variant, such that the biological activity of the functional variant is increased as compared to the parent TCR, polypeptide, or protein.

The TCR, polypeptide, or protein can consist essentially of the specified amino acid sequence or sequences described herein, such that other components of the TCR, polypeptide, or protein, e.g., other amino acids, do not materially change the biological activity of the TCR, polypeptide, or protein. In this regard, the inventive TCR, polypeptide, or protein can, for example, consist essentially of the amino acid sequence of SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, both of SEQ ID NOs: 23 and 24, both of SEQ ID NOs: 25 and 26, both of SEQ ID NOs: 27 and 28, or both of SEQ ID NOs: 29 and 30. Also, for instance, the inventive TCRs, polypeptides, or proteins can consist essentially of the amino acid sequence(s) of (i) SEQ ID NO: 7, (ii) SEQ ID NO: 8, (iii) SEQ ID NO: 9, (iv) SEQ ID NO: 10, (v) both of SEQ ID NOs: 7 and 8, or (vi) both of SEQ ID NOs: 9 and 10. Furthermore, the inventive TCRs, polypeptides, or proteins can consist essentially of the amino acid sequences of (a) all of SEQ ID NOs: 1-3, (b) all of SEQ ID NOs: 4-6, or (c) all of SEQ ID NOs: 1-6.

The TCRs, polypeptides, and proteins of the invention can be of any length, i.e., can comprise any number of amino acids, provided that the TCRs, polypeptides, or proteins retain their biological activity, e.g., the ability to specifically bind to G12V RAS; detect cancer in a mammal; or treat or prevent cancer in a mammal, etc. For example, the polypeptide can be in the range of from about 50 to about 5000 amino acids long, such as about 50, about 70, about 75, about 100, about 125, about 150, about 175, about 200, about 300, about 400, about 500, about 600, about 700, about 800, about 900, about 1000 or more amino acids in length. In this regard, the polypeptides of the invention also include oligopeptides.

The TCRs, polypeptides, and proteins of the invention can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α, γ-diaminobutyric acid, α, β-diaminopropionic acid, homophenylalanine, and α-tert-butylglycine.

The TCRs, polypeptides, and proteins of the invention can be glycosylated, amidated, carboxylated, phosphorylated, esterified, N-acylated, cyclized via, e.g., a disulfide bridge, or converted into an acid addition salt and/or optionally dimerized or polymerized, or conjugated.

The inventive TCRs, polypeptides, and proteins described herein (including any of the functional portions or variants thereof) are also contemplated to be useful as the soluble TCR component of bispecific engager TCR fusion proteins (e.g., IMMTAC (immune-mobilizing monoclonal TCRs against cancer) molecules). Bispecific engager TCR fusion proteins have two components. One component comprises a soluble TCR. The other component comprises an anti-CD3 effector. The anti-CD3 effector may be any molecule that engages with a CD3 molecule on T cells and activates a T cell immune response. For example, the anti-CD3 effector may be an anti-CD3 antibody or anti-CD3 antibody fragment. The soluble TCR component of the bispecific engager TCR fusion protein binds to the target antigen presented on the surface of cancer cells presented by an HLA molecule. The anti-CD3 effector component engages a CD3 molecule on T cells. The engagement of these components of the bispecific engager TCR fusion protein triggers the activation and recruitment of T cells and redirects T-cell killing to tumor cells. An aspect of the invention provides a bispecific engager TCR fusion protein comprising (i) any of the inventive TCRs, polypeptides, or proteins (including any of the functional portions or variants thereof) described herein and (ii) an anti-CD3 engager. Hereinafter, references to “protein(s)” also encompass the bispecific engager TCR fusion proteins described herein, unless specified otherwise.

The TCR, polypeptide, and/or protein of the invention can be obtained by methods known in the art such as, for example, de novo synthesis. Also, polypeptides and proteins can be recombinantly produced using the nucleic acids described herein using standard recombinant methods. See, for instance, Green and Sambrook, Molecular Cloning: A Laboratory Manual, 4^{th} ed., Cold Spring Harbor Press, Cold Spring Harbor, NY (2012). Alternatively, the TCRs, polypeptides, and/or proteins described herein can be synthesized by any of a variety of commercial entities. In this respect, the inventive TCRs, polypeptides, and proteins can be synthetic, recombinant, isolated, and/or purified. An aspect of the invention provides an isolated or purified TCR, polypeptide, or protein encoded by any of the nucleic acids or vectors described herein with respect to other aspects of the invention. Another aspect of the invention provides an isolated or purified TCR, polypeptide, or protein that results from expression of any of the nucleic acids or vectors described herein with respect to other aspects of the invention in a cell. Still another aspect of the invention provides a method of producing any of the TCRs, polypeptides, or proteins described herein, the method comprising culturing any of the host cells or populations of host cells described herein so that the TCR, polypeptide, or protein is produced.

Included in the scope of the invention are conjugates, e.g., bioconjugates, comprising any of the inventive TCRs, polypeptides, or proteins (including any of the functional portions or variants thereof), nucleic acids, recombinant expression vectors, host cells, populations of host cells, or antibodies, or antigen binding portions thereof. Conjugates, as well as methods of synthesizing conjugates in general, are known in the art.

An aspect of the invention provides a nucleic acid comprising a nucleotide sequence encoding any of the TCRs, polypeptides, proteins, or bispecific engager TCR fusion proteins described herein. “Nucleic acid,” as used herein, includes “polynucleotide,” “oligonucleotide,” and “nucleic acid molecule,” and generally means a polymer of DNA or RNA, which can be single-stranded or double-stranded, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In an aspect, the nucleic acid comprises complementary DNA (cDNA). It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

Preferably, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. See, for example, Green and Sambrook et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, β-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, β-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be synthesized by any of a variety of commercial entities.

The nucleic acid can comprise any nucleotide sequence which encodes any of the TCRs, polypeptides, or proteins described herein. In an aspect of the invention, the nucleic acid comprises a codon-optimized nucleotide sequence encoding any of the TCRs, polypeptides, or proteins described herein. Without being bound to any particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency.

An aspect of the invention provides an isolated or purified nucleic acid comprising, from 5′ to 3′, a first nucleic acid sequence and a second nucleotide sequence, wherein the first and second nucleotide sequence, respectively, encode the amino sequences of SEQ ID NOs: 7 and 8; 8 and 7; 9 and 10; 10 and 9; 23 and 24; 24 and 23; 25 and 26; 26 and 25; 27 and 28; 28 and 27; 29 and 30; or 30 and 29.

In an aspect of the invention, the isolated or purified nucleic acid further comprises a third nucleotide sequence interposed between the first and second nucleotide sequence, wherein the third nucleotide sequence encodes a cleavable linker peptide. In an aspect of the invention, the cleavable linker peptide comprises the amino acid sequence of RAKRSGSGATNFSLLKQAGDVEENPGP (SEQ ID NO: 31).

The nucleic acids of the invention can be incorporated into a recombinant expression vector. In this regard, the invention provides a recombinant expression vector comprising any of the nucleic acids of the invention. In an aspect of the invention, the recombinant expression vector comprises a nucleotide sequence encoding the a chain, the β chain, and linker peptide.

For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotide, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages do not hinder the transcription or replication of the vector.

The recombinant expression vector of the invention can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as λGT10, λGT11, λZapII (Stratagene), λEMBL4, and λNM1149, also can be used. Examples of plant expression vectors include pBIO1, pBI101.2, pBI101.3, pBI121 and pBIN19 (Clontech). Examples of animal expression vectors include pEUK-Cl, pMAM and pMAMneo (Clontech). Preferably, the recombinant expression vector is a viral vector, e.g., a retroviral vector. In an especially preferred aspect, the recombinant expression vector is an MSGV1 vector. In an aspect of the invention, the recombinant expression vector is a transposon or a lentiviral vector.

The recombinant expression vectors of the invention can be prepared using standard recombinant DNA techniques described in, for example, Green and Sambrook et al., supra. Constructs of expression vectors, which are circular or linear, can be prepared to contain a replication system functional in a prokaryotic or eukaryotic host cell. Replication systems can be derived, e.g., from ColE1, 2 μ plasmid, λ, SV40, bovine papillomavirus, and the like.

Desirably, the recombinant expression vector comprises regulatory sequences, such as transcription and translation initiation and termination codons, which are specific to the type of host cell (e.g., bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected host cells. Marker genes include biocide resistance, e.g., resistance to antibiotics, heavy metals, etc., complementation in an auxotrophic host cell to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the TCR, polypeptide, or protein. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, nitroreductase, and the inducible caspase 9 gene system.

Another aspect of the invention further provides a host cell comprising any of the nucleic acids or recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell is preferably a prokaryotic cell, e.g., a DH5α cell. For purposes of producing a recombinant TCR, polypeptide, or protein, the host cell is preferably a mammalian cell. Most preferably, the host cell is a human cell. While the host cell can be of any cell type, can originate from any type of tissue, and can be of any developmental stage, the host cell preferably is a peripheral blood lymphocyte (PBL) or a peripheral blood mononuclear cell (PBMC). More preferably, the host cell is a T cell. In an aspect of the invention, the host cell is a human lymphocyte. In another aspect of the invention, the host cell is selected from the group consisting of a T cell, a natural killer T (NKT) cell, an invariant natural killer T (iNKT) cell, a natural killer (NK) cell, a macrophage, a pluripotent cell, and a multipotent cell. Still another aspect of the invention provides a method of producing a host cell expressing a TCR that has antigenic specificity for the peptide of VVVGAVGVGK (SEQ ID NO: 33), the method comprising contacting a cell with any of the vectors described herein under conditions that allow introduction of the vector into the cell.

For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. Preferably, the T cell is a human T cell. The T cell can be any type of T cell and can be of any developmental stage, including but not limited to, CD4⁺/CD8⁺ double positive T cells, CD4⁺ helper T cells, e.g., Th₁ and Th₂ cells, CD4⁺ T cells, CD8⁺ T cells (e.g., cytotoxic T cells), tumor infiltrating lymphocytes (TILs), memory T cells (e.g., central memory T cells and effector memory T cells), naïve T cells, and the like.

In an aspect of the invention, the host cell is a pluripotent cell or a multipotent cell. Pluripotent cells have the capacity to give rise to any of the three germ layers: endoderm, mesoderm, and ectoderm. Pluripotent cells may comprise, for example, stem cells, e.g., embryonic stem cells, nuclear transfer derived embryonic stem cells, induced pluripotent stem cells (iPSC), etc. Multipotent cells may comprise, for example, hematopoietic stem cells. Modifying, e.g., reprogramming, cells to a pluripotent state refers to the reversion of a cell to a pluripotent cell and is described for example, in Crompton et al., Trends Immunol., 35(4): 178-185 (2014). Exemplary techniques may include somatic cell nuclear transfer (SCNT), cell-cell fusion, and direct reprogramming. Examples of methods for carrying out cell-cell fusion are described, for example, in Ogle et al., Nat. Rev. Mol. Cell Biol. 6; 567-75 (2005) and Zhou et al., Cell Stem Cell, 3; 382-388 (2008). Examples of methods for carrying out SCNT are described, for example, in Hanna et al., Cell, 143: 508-525 (2010); Stadtfeld et al., Genes Dev., 24: 2239-2263 (2010); Wilmut et al., Nature, 385: 810-813 (1997); Vizcardo et al., Cell Stem Cell, 12: 31-36 (2013); and Crompton et al., Cell Stem Cell, 12: 6-8 (2013). In an aspect of the invention, the host cell is an iPSC that was prepared by reprogramming any of the host cells described herein (e.g., T cells, NK cells, or invariant natural killer T cells) to a pluripotent state.

Also provided by the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cells, a muscle cell, a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one aspect of the invention, the population of cells is a clonal population comprising host cells comprising a recombinant expression vector as described herein.

In an aspect of the invention, the numbers of cells in the population may be rapidly expanded. Expansion of the numbers of T cells can be accomplished by any of a number of methods as are known in the art as described in, for example, U.S. Pat. Nos. 8,034,334; 8,383,099; U.S. Patent Application Publication No. 2012/0244133; Dudley et al., J. Immunother., 26:332-42 (2003); and Riddell et al., J. Immunol. Methods, 128:189-201 (1990). In an aspect, expansion of the numbers of T cells is carried out by culturing the T cells with OKT3 antibody, IL-2, and feeder PBMC (e.g., irradiated allogeneic PBMC).

The inventive TCRs, polypeptides, proteins, nucleic acids, recombinant expression vectors, and host cells (including populations thereof), can be isolated and/or purified. The term “isolated,” as used herein, means having been removed from its natural environment. The term “purified,” as used herein, means having been increased in purity, wherein “purity” is a relative term, and not to be necessarily construed as absolute purity. For example, the purity can be at least about 50%, can be greater than about 60%, about 70%, about 80%, about 90%, about 95%, or can be about 100%.

The inventive TCRs, polypeptides, proteins, bispecific engager TCR fusion proteins, nucleic acids, recombinant expression vectors, and host cells (including populations thereof), all of which are collectively referred to as “inventive TCR materials” hereinafter, can be formulated into a composition, such as a pharmaceutical composition. In this regard, the invention provides a pharmaceutical composition comprising any of the TCRs, polypeptides, proteins, bispecific engager TCR fusion proteins, nucleic acids, expression vectors, or host cells (including populations thereof), described herein, and a pharmaceutically acceptable carrier. The inventive pharmaceutical compositions containing any of the inventive TCR materials can comprise more than one inventive TCR material, e.g., a polypeptide and a nucleic acid, or two or more different TCRs. Alternatively, the pharmaceutical composition can comprise an inventive TCR material in combination with another pharmaceutically active agent(s) or drug(s), such as a chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

Preferably, the carrier is a pharmaceutically acceptable carrier. With respect to pharmaceutical compositions, the carrier can be any of those conventionally used for the particular inventive TCR material under consideration. Methods for preparing administrable compositions are known or apparent to those skilled in the art and are described in more detail in, for example, Remington: The Science and Practice of Pharmacy, 22^{nd} Ed., Pharmaceutical Press (2012). It is preferred that the pharmaceutically acceptable carrier be one which has no detrimental side effects or toxicity under the conditions of use.

The choice of carrier will be determined in part by the particular inventive TCR material, as well as by the particular method used to administer the inventive TCR material. Accordingly, there are a variety of suitable formulations of the pharmaceutical composition of the invention. Suitable formulations may include any of those for parenteral, subcutaneous, intravenous, intramuscular, intraarterial, intrathecal, intratumoral, or interperitoneal administration. More than one route can be used to administer the inventive TCR materials, and in certain instances, a particular route can provide a more immediate and more effective response than another route.

Preferably, the inventive TCR material is administered by injection, e.g., intravenously. When the inventive TCR material is a host cell (or population thereof) expressing the inventive TCR, the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In an aspect, the pharmaceutically acceptable carrier is supplemented with human serum albumen.

For purposes of the invention, the amount or dose (e.g., numbers of cells when the inventive TCR material is one or more cells) of the inventive TCR material administered should be sufficient to effect, e.g., a therapeutic or prophylactic response, in the subject or animal over a reasonable time frame. For example, the dose of the inventive TCR material should be sufficient to bind to a cancer antigen (e.g., G12V RAS), or detect, treat or prevent cancer in a period of from about 2 hours or longer, e.g., 12 to 24 or more hours, from the time of administration. In certain aspects, the time period could be even longer. The dose will be determined by the efficacy of the particular inventive TCR material and the condition of the animal (e.g., human), as well as the body weight of the animal (e.g., human) to be treated.

Many assays for determining an administered dose are known in the art. For purposes of the invention, an assay, which comprises comparing the extent to which target cells are lysed or IFN-γ is secreted by T cells expressing the inventive TCR, polypeptide, or protein upon administration of a given dose of such T cells to a mammal among a set of mammals of which each is given a different dose of the T cells, could be used to determine a starting dose to be administered to a mammal. The extent to which target cells are lysed or IFN-γ is secreted upon administration of a certain dose can be assayed by methods known in the art.

The dose of the inventive TCR material also will be determined by the existence, nature and extent of any adverse side effects that might accompany the administration of a particular inventive TCR material. Typically, the attending physician will decide the dosage of the inventive TCR material with which to treat each individual patient, taking into consideration a variety of factors, such as age, body weight, general health, diet, sex, inventive TCR material to be administered, route of administration, and the severity of the cancer being treated. In an aspect in which the inventive TCR material is a population of cells, the number of cells administered per infusion may vary, e.g., from about 1×10⁶ to about 1×10¹² cells or more. In certain aspects, fewer than 1×10⁶ cells may be administered.

One of ordinary skill in the art will readily appreciate that the inventive TCR materials of the invention can be modified in any number of ways, such that the therapeutic or prophylactic efficacy of the inventive TCR materials is increased through the modification. For instance, the inventive TCR materials can be conjugated either directly or indirectly through a bridge to a chemotherapeutic agent. The practice of conjugating compounds to a chemotherapeutic agent is known in the art. One of ordinary skill in the art recognizes that sites on the inventive TCR materials, which are not necessary for the function of the inventive TCR materials, are suitable sites for attaching a bridge and/or a chemotherapeutic agent, provided that the bridge and/or chemotherapeutic agent, once attached to the inventive TCR materials, do(es) not interfere with the function of the inventive TCR materials, i.e., the ability to bind to G12V RAS or to detect, treat, or prevent cancer.

It is contemplated that the inventive pharmaceutical compositions, TCRs, polypeptides, proteins, bispecific engager TCR fusion proteins, nucleic acids, recombinant expression vectors, host cells, and populations of cells can be used in methods of treating or preventing cancer. Without being bound to a particular theory, the inventive TCRs are believed to bind specifically to G12V RAS, such that the TCR (or related inventive polypeptide or protein), when expressed by a cell, is able to mediate an immune response against a target cell expressing G12V RAS. In this regard, an aspect of the invention provides a method of treating or preventing cancer in a mammal, comprising administering to the mammal any of the pharmaceutical compositions, TCRs, polypeptides, proteins, or bispecific engager TCR fusion proteins described herein, any nucleic acid or recombinant expression vector comprising a nucleotide sequence encoding any of the TCRs, polypeptides, proteins, or bispecific engager TCR fusion proteins described herein, or any host cell or population of cells comprising a recombinant vector which encodes any of the TCRs, polypeptides, proteins, or bispecific engager TCR fusion proteins described herein, in an amount effective to treat or prevent cancer in the mammal.

An aspect of the invention provides a method of inducing an immune response against a cancer in a mammal, comprising administering to the mammal any of the pharmaceutical compositions, TCRs, polypeptides, proteins, or bispecific engager TCR fusion proteins described herein, any nucleic acid or recombinant expression vector comprising a nucleotide sequence encoding any of the TCRs, polypeptides, proteins, or bispecific engager TCR fusion proteins described herein, or any host cell or population of cells comprising a recombinant vector which encodes any of the TCRs, polypeptides, proteins, or bispecific engager TCR fusion proteins described herein, in an amount effective to induce an immune response against the cancer in the mammal.

An aspect of the invention provides any of the pharmaceutical compositions, TCRs, polypeptides, proteins, or bispecific engager TCR fusion proteins described herein, any nucleic acid or recombinant expression vector comprising a nucleotide sequence encoding any of the TCRs, polypeptides, proteins, or bispecific engager TCR fusion proteins described herein, or any host cell or population of cells comprising a recombinant vector which encodes any of the TCRs, polypeptides, proteins, or bispecific engager TCR fusion proteins described herein, for use in the treatment or prevention of cancer in a mammal.

An aspect of the invention provides any of the pharmaceutical compositions, TCRs, polypeptides, proteins, or bispecific engager TCR fusion proteins described herein, any nucleic acid or recombinant expression vector comprising a nucleotide sequence encoding any of the TCRs, polypeptides, proteins, or bispecific engager TCR fusion proteins described herein, or any host cell or population of cells comprising a recombinant vector which encodes any of the TCRs, polypeptides, proteins, or bispecific engager TCR fusion proteins described herein, for use in inducing an immune response against a cancer in a mammal.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the cancer being treated or prevented. For example, treatment or prevention can include promoting the regression of a tumor. Also, for purposes herein, “prevention” can encompass delaying the onset of the cancer, or a symptom or condition thereof. Alternatively or additionally, “prevention” may encompass preventing or delaying the recurrence of cancer, or a symptom or condition thereof.

Also provided is a method of detecting the presence of cancer in a mammal. The method comprises (i) contacting a sample comprising one or more cells from the mammal with any of the inventive TCRs, polypeptides, proteins, bispecific engager TCR fusion proteins, nucleic acids, recombinant expression vectors, host cells, populations of cells, or pharmaceutical compositions described herein, thereby forming a complex, and (ii) detecting the complex, wherein detection of the complex is indicative of the presence of cancer in the mammal.

With respect to the inventive method of detecting cancer in a mammal, the sample of cells can be a sample comprising whole cells, lysates thereof, or a fraction of the whole cell lysates, e.g., a nuclear or cytoplasmic fraction, a whole protein fraction, or a nucleic acid fraction.

For purposes of the inventive method of detecting cancer, the contacting can take place in vitro or in vivo with respect to the mammal. Preferably, the contacting is in vitro.

Also, detection of the complex can occur through any number of ways known in the art. For instance, the inventive TCRs, polypeptides, proteins, nucleic acids, recombinant expression vectors, host cells, or populations of cells, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

For purposes of the inventive methods, wherein host cells or populations of cells are administered, the cells can be cells that are allogeneic or autologous to the mammal. Preferably, the cells are autologous to the mammal.

With respect to the inventive methods, the cancer can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, colorectal cancer, endometrial cancer, esophageal cancer, uterine cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer. A preferred cancer is pancreatic, colorectal, lung, endometrial, ovarian, or prostate cancer. Preferably, the lung cancer is lung adenocarcinoma, the ovarian cancer is epithelial ovarian cancer, and the pancreatic cancer is pancreatic adenocarcinoma. In an aspect of the invention, the cancer expresses a mutated human RAS amino acid sequence with a substitution of glycine at position 12 with valine, wherein the mutated human RAS amino acid sequence is a mutated human KRAS, a mutated human HRAS, or a mutated human NRAS amino acid sequence, and wherein position 12 is defined by reference to the WT human KRAS, WT human HRAS, or WT human NRAS protein, respectively. The mutated human KRAS, mutated human HRAS, and mutated human NRAS expressed by the cancer may be as described herein with respect to other aspects of the invention.

The mammal referred to in the inventive methods can be any mammal. As used herein, the term “mammal” refers to any mammal, including, but not limited to, mammals of the order Rodentia, such as mice and hamsters, and mammals of the order Lagomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perissodactyla, including Equines (horses). It is most preferred that the mammals are of the order Primates or Ceboids (monkeys) or of the order Anthropoids (humans and apes). An especially preferred mammal is the human.

The following examples further illustrate the invention but, of course, should not be construed as in any way limiting its scope.

### EXAMPLE 1

This example demonstrates the isolation of a TCR having antigenic specificity for G12V RAS presented by HLA-A3.

A 10-mer G12V RAS peptide (VVVGAVGVGK) (SEQ ID NO: 33) was detected on HLA-A0301 complexes using mass spectrometric analysis. This peptide (and a helper peptide) were used to immunize HLA-A0301 transgenic mice to obtain mouse T cells that can recognize G12V RAS presented by HLA-A0301.

The spleens and draining lymph nodes (DLNs) of the immunized mice were stimulated in vitro (IVS) with the 10-mer peptide. The reactivities of the splenocytes and DLNs were assessed using IFN-gamma ELISA. Single cells were sorted based on tetramer staining. One TCR (“TCR 807”) having antigenic specificity for G12V RAS presented by HLA-A3 was identified and isolated from the draining lymph nodes of immunized mice. The TCR alpha and beta chains of the isolated T-cells were sequenced. The TCR alpha and beta chains were cloned and pairs of alpha and beta chains were tested against target cell lines using IFN-gamma ELISA assays.

The WT amino acid sequences of the isolated, full-length TCR alpha and beta chains are set forth in Table 5. In Table 5, the N-terminal signal peptides are in bold font, the CDRs are underlined, and the constant regions are italicized.

TABLE 5

| | Amino acid sequences (N- terminus-CDR1, CDR2, |
|---|---|
| TCR chain | CDR3-C terminus) |
| Alpha chain | MKSLSVLLVVLWLQLNCVRSQQKVQQSPESLSVPE |
| (mouse TRAV7D-4*02, | GGMASLNCTSSDRNFQYFWWYRQHSGEGPKALMSIF |
| TRAJ5*01) | SDGDKKEGRFTAHLNKASLHVSLHIRDSQPSDSALYF |
| | CAARPGTQVVGQLTFGRGTRLQVYANIQNPEPAVYQL |
| | KDPRSQDSTLCLFTDFDSQINVPKTMESGTFITDKTVLD |
| | MKAMDSKSNGAIAWSNQTSFTCQDIFKETNATYPSSDVP |
| | CDATLTEKSFETDMNLNFQNLSVMGLRILLLKVAGENLL |
| | MTLRLWSS (SEQ ID NO: 27) |
| Beta chain | MALYSLLAFLLGMFLGVSAQTIHQWPVAEIKAVGSP |
| (mouse TRBV31*01, | LSLGCTIKGKSSPNLYWYWQATGGTLQQLFYSITVGQ |
| TRBJ2-7*01, TRBD1*01) | VESVVQLNLSASRPKDDQFILSTEKLLLSHSGFYLCAW |
| | RDRPSSYEQYFGPGTRLTVLEDLRNVTPPKVSLFEPSKA |
| | ELANKOKATLVCLARGFFPDHVELSWWVNGKEVHSGVST |
| | DPQAYKESNYSYCLSSRLRVSATFWHNPRNHFRCQVQFH |
| | GLSEEDKWPEGSPKPVTQNISAEAWGRADCGITSASYHQ |
| | GVLSATILYEILLGKATLYAVLVSGLVLMAMVKKKNS |
| | (SEQ ID NO: 28) |

### EXAMPLE 2

This example demonstrates the preparation of retroviral vectors encoding the TCR of Example 1.

The nucleotide sequences encoding the TCR alpha and beta chains of Table 5 were codon-optimized. The codon-optimized TCR alpha and beta chain sequences were cloned into the MSGV1 retroviral vector. A furin/SGSG/P2A linker having the amino acid sequence of RAKRSGSGATNFSLLKQAGDVEENPGP (SEQ ID NO: 31) was positioned between the alpha and beta chains. The configuration of the retroviral vector was as follows: amino terminus (N)-beta chain-linker-alpha chain-carboxyl terminus (C).

### EXAMPLE 3

This example demonstrates that human T cells transduced with the retroviral vector of Example 2 recognize tumor cell lines that express both HLA-A3 and G12V RAS.

PBL from healthy human donors (Healthy Donor #1 (HD1) and Healthy Donor #2 (HD2) were transduced with the retroviral vector of Example 2 (Tables 6A-6B) or an empty vector (mock-transduced cells) (“mock”) (control) (Tables 6C-6D). Transduced cells (effector cells) were co-cultured with one of the following target tumor cell lines: 4393 (A3+, G12V−), 4385 (A3−G12V+), 4316 (A3+, G12V+), 4395 (A3+, G12V+), or 4391 (A3+, G12V+). In this Example, “G12V+” indicates that the tumor cell line naturally expresses G12V RAS, “G12V−” indicates that the tumor cell line does not naturally express G12V RAS, “A3+” indicates that the tumor cell line naturally expresses HLA-A3, and “A3−” indicates that the tumor cell line does not naturally express HLA-A3. The target tumor cell lines were either (i) pulsed with VVVGAVGVGK (SEQ ID NO: 33) (positive control) or VVVGAGGVGK (SEQ ID NO: 34) (negative control) or (ii) not pulsed with any mutated or WT RAS peptide. Healthy donor (HD) T cells cultured alone served as a control. IFN-γ was measured. The results are shown in Tables 6A-6D. Tables 6A-6D show the concentration of IFN-gamma measured following the co-culture. In Tables 6A-6D, the “media” columns provide the results obtained following co-culture with target cells that were not pulsed with any mutated or WT RAS peptide, and the columns labeled with the RAS peptides indicate the results obtained following co-culture with target cells that were pulsed with the indicated RAS peptide.

As shown in Tables 6A-6D, human T cells transduced with the retroviral vector of Example 2 specifically recognized tumor cells that express both HLA-A3 and G12V RAS, but not tumor cells which lacked expression of HLA-A3 or G12V RAS.

TABLE 6A

| HD1/G12V/A3 mTCR_807 | HLA-A3- | | IFNg = pg/ml |
|---|---|---|---|
| | media | VVVGAVGVGK | VVVGAGGVGK |
| | | SEQ ID NO: 33 | SEQ ID NO: 34 |
| HD1 T cells alone | 0 | 0 | 0 |
| 4393 (A3+, G12V−) | 0 | 2661 | 0 |
| 4385 (A3− G12V+) | 0 | 0 | 0 |
| 4316 (A3+, G12V+) | 4953 | 22729 | 4173 |
| 4395 (A3+, G12V+) | 0 | 6704 | 0 |
| 4391 (A3+, G12V+) | 6 26 | 8941 | 461 |

TABLE 6B

| HD2/G12V/A3 mTCR_807 | HLA-A3- | | IFNg = pg/ml |
|---|---|---|---|
| | media | VVVGAVGVGK | VVVGAGGVGK |
| | | SEQ ID NO: 33 | SEQ ID NO: 34 |
| HD2 T cells alone | 0 | 0 | 0 |
| 4393 (A3+, G12V−) | 0 | 2027 | 0 |
| 4385 (A3− G12V+) | 0 | 0 | 0 |
| 4316 (A3+, G12V+) | 4034 | 14447 | 3080 |
| 4395 (A3+, G12V+) | 0 | 5921 | 0 |
| 4391 (A3+, G12V+) | 849 | 7094 | 662 |

TABLE 6C

| HD1/mock | HLA-A3- | | IFNg = pg/ml |
|---|---|---|---|
| | media | VVVGAVGVGK | VVVGAGGVGK |
| | | SEQ ID NO: 33 | SEQ ID NO: 34 |
| HD1 T cells alone | 0 | 0 | 0 |
| 4393 (A3+, G12V−) | 0 | 0 | 0 |
| 4385 (A3− G12V+) | 0 | 0 | 0 |
| 4316 (A3+, G12V+) | 0 | 0 | 0 |
| 4395 (A3+, G12V+) | 0 | 0 | 0 |
| 4391 (A3+, G12V+) | 0 | 0 | 0 |

TABLE 6D

| HD2/mock | HLA-A3- | | IFNg = pg/ml |
|---|---|---|---|
| | media | VVVGAVGVGK | VVVGAGGVGK |
| | | SEQ ID NO: 33 | SEQ ID NO: 34 |
| HD2 T cells alone | 0 | 0 | 0 |
| 4393 (A3+, G12V−) | 0 | 0 | 0 |
| 4385 (A3− G12V+) | 0 | 0 | 0 |
| 4316 (A3+, G12V+) | 159 | 0 | 0 |
| 4395 (A3+, G12V+) | 0 | 0 | 0 |
| 4391 (A3+, G12V+) | 0 | 0 | 0 |

### EXAMPLE 4

This example demonstrates the anti-tumor activity of human T cells transduced with the retroviral vector of Example 2 in a mouse bearing human tumors.

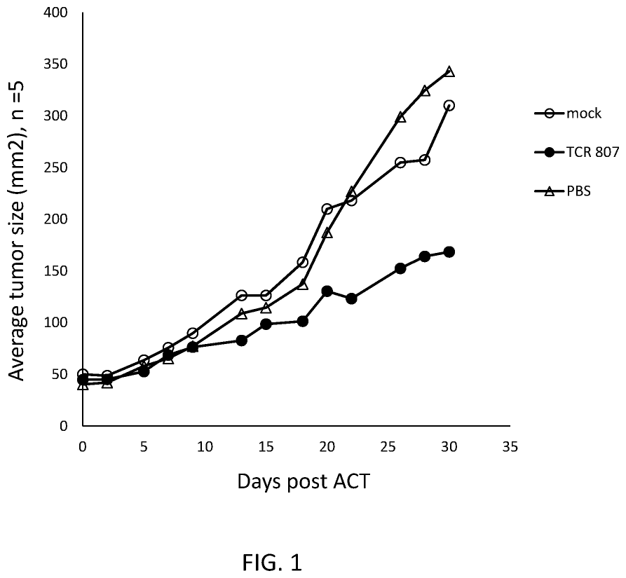

Human tumor cells 4391 (A3+, G12V+) were implanted into mice to produce tumor-bearing mice. Mice were treated with human T cells transduced with the retroviral vector of Example 2, phosphate-buffered saline (PBS) (control), or human T cells transduced with an empty vector (mock-transduced cells) (control). The size of the tumors was measured at various time points following administration of the transduced cells to the mice (adoptive cell therapy (ACT)). The results are shown in FIG. 1. As shown in FIG. 1, the size of the tumors in the mice treated with T cells transduced with the retroviral vector of Example 2 was diminished as compared to the size of the tumors in the mice treated with PBS or mock-transduced cells.

### EXAMPLE 5

This example demonstrates that the TCR encoded by the retroviral vector of Example 2 demonstrates higher avidity to mutant peptide pulsed target cells as compared to a previously identified TCR.

A TCR having antigenic specificity for G12V RAS presented by HLA-A3 was previously identified, isolated, and disclosed in U.S. Patent Application Publication No. 2020/0129555 (“hereinafter, TCR 1”). The avidity of this prior TCR 1 was compared to that of the TCR encoded by the retroviral vector of Example 2 of the present patent application (TCR 807).

PBL from healthy human donors were transduced with a retroviral vector encoding TCR 1 or TCR 807. PBL transduced with an empty vector (mock) served as a control.

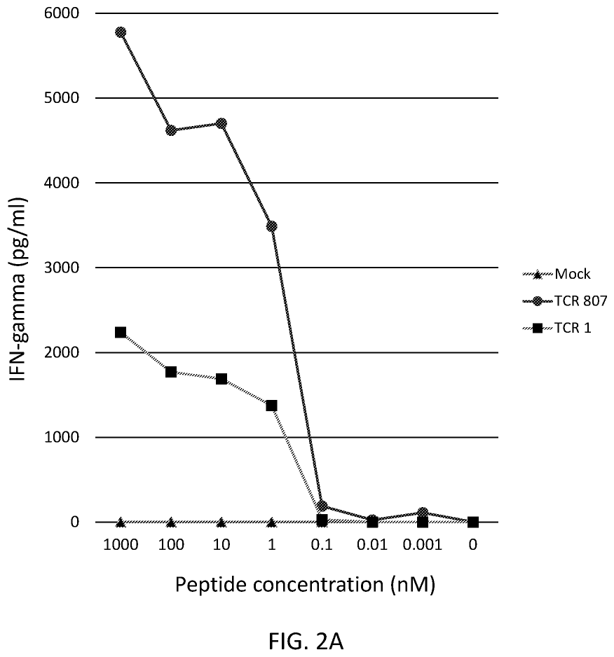

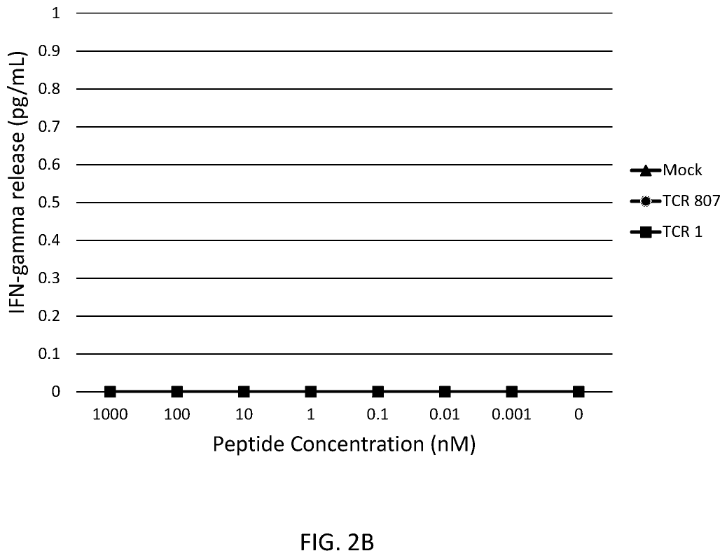

Cos7 cells transduced with human HLA A*03:01 were independently pulsed with the G12V RAS peptide VVVGAVGVGK (SEQ ID NO: 33) or the corresponding wild-type peptide VVVGAGGVGK (WT) (SEQ ID NO: 34) at the concentrations shown in FIGS. 2A-2B. The transduced cells (effector cells) were co-cultured with the peptide-pulsed target cells. Interferon-gamma secretion was measured. The results are shown in FIGS. 2A-2B.

As shown in FIGS. 2A-2B, TCR 807-transduced cells demonstrated avid recognition of mutated peptide-pulsed target cells at a pulsed peptide concentration as low as 0.1 nM. In contrast, TCR 1 transduced cells recognized mutated peptide-pulsed target cells at a pulsed peptide concentration as low as 1 nM (FIG. 2A). Neither TCR demonstrated reactivity to WT peptide (FIG. 2B). Accordingly, the TCR 807 showed greater reactivity to the same target epitope as the prior TCR (TCR 1), while maintaining complete specificity for the mutated epitope.

### EXAMPLE 6

This example demonstrates that the TCR encoded by the retroviral vector of Example 2 demonstrates better reactivity and specificity to target cells as compared to a previously identified TCR.

PBL from healthy human donors were transduced with a retroviral vector encoding the TCR 1 described in Example 5 or the TCR encoded by the retroviral vector of Example 2 (TCR 807). PBL transduced with an empty vector (mock) served as a control.

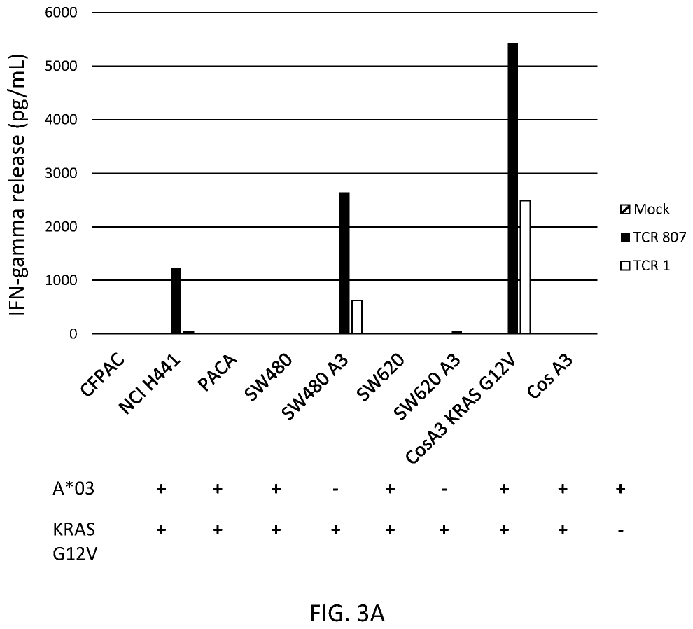

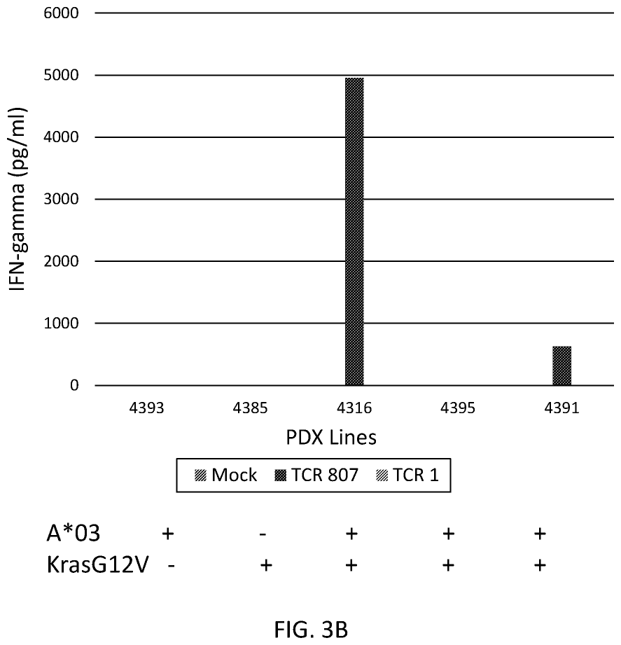

Target cells included (i) Cos cells transfected with HLA-A3 alone or both HLA-A3 and G12V RAS (FIG. 3A); (ii) RAS G12V positive tumor cell lines CFPAC, NCI H441, PACA, SW480, or SW620 that were either untransfected or transfected with HLA-A3, (FIG. 3A); or (iii) genetically unmodified patient-derived xenograft (PDX) tumor cell lines (4393, 4385, 4316, 4395, or 4391) (FIG. 3B). TCR-transduced cells (effector cells) were co-cultured with the target cells. Interferon-gamma secretion was measured. The results are shown in FIGS. 3A-3B.

TCR 807 demonstrated higher recognition of target cells transfected with HLA-A3 and G12V RAS as compared to TCR 1 (FIG. 3A). TCR 807 recognized 3 of 7 genetically unmodified tumor lines (NCI H441, 4316 and 4391), whereas TCR 1 was unable to recognize any genetically unmodified tumor lines (FIG. 3A). TCR 807 was able to recognize 2 of 2 HLA-A3 and RAS G12V positive PDX lines, while TCR 1 was not able to recognize either (FIG. 3B).

### EXAMPLE 7

This example demonstrates that human PBL transduced with a retroviral vector encoding TCR 807 kill an HLA-A*03:01+ colorectal cancer cell line with the G12V RAS mutation.

PBL from healthy human donors were transduced with a retroviral vector encoding the TCR 1 described in Example 5 or the TCR encoded by the retroviral vector of Example 2 (TCR 807). Human PBL transduced with an empty vector (mock) served as a control.

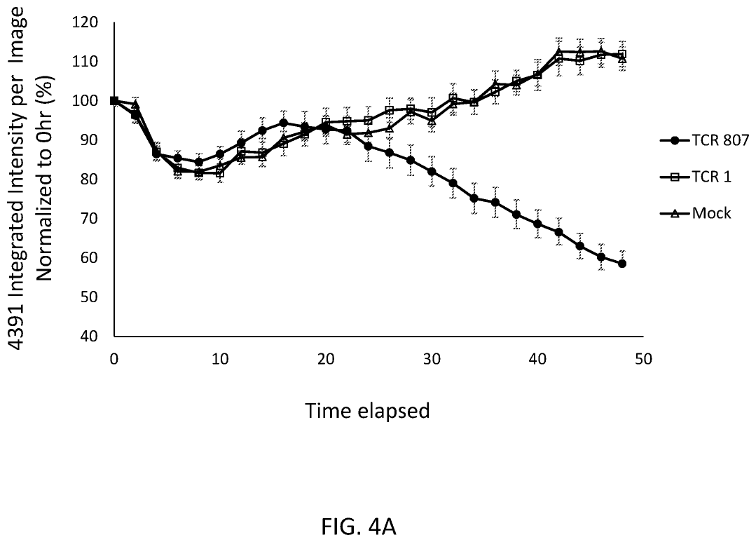

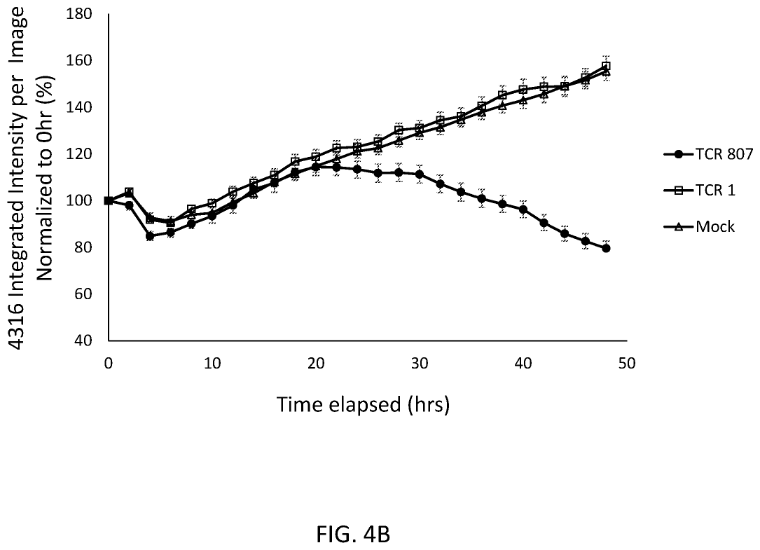

Transduced cells (effector cells) were co-cultured with tumor cell line 4391 or tumor cell line 4316. Cytotoxicity was measured over the course of the 48 hour co-culture and was evidenced by a reduction in the area covered by viable target cells using the INCUCYTE live-cell analysis system. The results are shown in FIGS. 4A-4B. The results showed that greater cytotoxicity was achieved with TCR 807 as compared to TCR 1.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising.” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated or purified T-cell receptor (TCR) comprising the amino acid sequences of:
(a) all of SEQ ID NOs: 1-3,
(b) all of SEQ ID NOs: 4-6, or
(c) all of SEQ ID NOs: 1-6,
wherein the TCR has antigenic specificity for a mutated human RAS amino acid sequence with a substitution of aspartic acid at position 12 with valine,
wherein the mutated human RAS amino acid sequence is a mutated human Kirsten rat sarcoma viral oncogene homolog (KRAS), a mutated human Harvey rat sarcoma viral oncogene homolog (HRAS), or a mutated human Neuroblastoma rat sarcoma viral oncogene homolog (NRAS) amino acid sequence, and
wherein position 12 is defined by reference to the wild-type human KRAS, wild-type human HRAS, or wild-type human NRAS protein, respectively.

2-4. (canceled).

5. The TCR according to claim 1, comprising the amino acid sequence(s) of:
(i) SEQ ID NO: 7,
(ii) SEQ ID NO: 8,
(iii) SEQ ID NO: 9,
(iv) SEQ ID NO: 10,
(v) both of SEQ ID NOs: 7 and 8,
(vi) both of SEQ ID NOs: 9 and 10;
(vii) SEQ ID NO: 23, wherein:
(i) X at position 181 of SEQ ID NO: 23 is Thr or Cys;
(ii) X at position 245 of SEQ ID NO: 23 is Ser, Ala, Val, Leu, Ile, Pro, Phe, Met, or Trp;
(iii) X at position 247 of SEQ ID NO: 23 is Met, Ala, Val, Leu, Ile, Pro, Phe, or Trp; and
(iv) X at position 248 of SEQ ID NO: 23 is Gly, Ala, Val, Leu, Ile. Pro, Phe, Met, or Trp;
(viii) SEQ ID NO: 24, wherein X at position 188 of SEQ ID NO: 24 is Ser or Cys;
(ix) both (vii) and (viii);
(x) SEQ ID NO: 25, wherein:
(i) X at position 161 of SEQ ID NO: 25 is Thr or Cys;
(ii) X at position 225 of SEQ ID NO: 25 is Ser, Ala, Val, Leu, Ile, Pro, Phe, Met, or Trp;
(iii) X at position 227 of SEQ ID NO: 25 is Met, Ala, Val, Leu, Ile, Pro, Phe, or Trp; and
(iv) X at position 228 of SEQ ID NO: 25 is Gly, Ala, Val, Leu, Ile, Pro, Phe, Met, or Trp;
(xi) SEQ ID NO: 26, wherein X at position 170 of SEQ ID NO: 26 is Ser or Cys;
(xii) both (x) and (xi);
(xiii) SEQ ID NO: 27;
(xiv) SEQ ID NO: 28;
(xv) SEQ ID NO: 29;
(xvi) SEQ ID NO: 30;
(xvii) both (xiii) and (xiv); or
(xviii) both (xv) and (xvi).

6. The TCR of claim 1, further comprising:
(a) an α chain constant region comprising the amino acid sequence of SEQ ID NO: 19, wherein:
(i) X at position 48 of SEQ ID NO: 19 is Thr or Cys;
(ii) X at position 112 of SEQ ID NO: 19 is Ser, Ala, Val, Leu, Ile, Pro, Phe, Met, or Trp;
(iii) X at position 114 of SEQ ID NO: 19 is Met, Ala, Val, Leu, Ile, Pro, Phe, or Trp; and
(iv) X at position 115 of SEQ ID NO: 19 is Gly, Ala, Val, Leu, Ile, Pro, Phe, Met, or Trp;
(b) a β chain constant region comprising the amino acid sequence of SEQ ID NO: 20, wherein X at position 57 of SEQ ID NO: 20 is Ser or Cys; or
(c) both (a) and (b).

7. (canceled).

8. An isolated or purified polypeptide comprising a functional portion of the TCR of claim 1, wherein the functional portion comprises the amino acid sequences of:
(a) all of SEQ ID NOs: 1-3,
(b) all of SEQ ID NOs: 4-6, or
(c) all of SEQ ID NOs: 1-6.

9. The isolated or purified polypeptide according to claim 8, wherein the functional portion comprises the amino acid sequence(s) of:
(i) SEQ ID NO: 7,
(ii) SEQ ID NO: 8,
(iii) SEQ ID NO: 9,
(iv) SEQ ID NO: 10,
(v) both of SEQ ID NOs: 7 and 8,
(vi) both of SEQ ID NOs: 9 and 10;
(vii) SEQ ID NO: 23, wherein:
(i) X at position 181 of SEQ ID NO: 23 is Thr or Cys;
(ii) X at position 245 of SEQ ID NO: 23 is Ser, Ala, Val, Leu, Ile, Pro, Phe, Met, or Trp;
(iii) X at position 247 of SEQ ID NO: 23 is Met, Ala, Val, Leu, Ile, Pro, Phe, or Trp; and
(iv) X at position 248 of SEQ ID NO: 23 is Gly, Ala, Val, Leu, Ile, Pro, Phe, Met, or Trp;
(viii) SEQ ID NO: 24, wherein X at position 188 of SEQ ID NO: 24 is Ser or Cys;
(ix) both (vii) and (viii);
(x) SEQ ID NO: 25, wherein:
(i) X at position 161 of SEQ ID NO: 25 is Thr or Cys;
(ii) X at position 225 of SEQ ID NO: 25 is Ser, Ala, Val, Leu, Ile, Pro, Phe, Met, or Trp;
(iii) X at position 227 of SEQ ID NO: 25 is Met, Ala, Val, Leu, Ile, Pro, Phe, or Trp; and
(iv) X at position 228 of SEQ ID NO: 25 is Gly, Ala, Val, Leu, Ile, Pro, Phe, Met, or Trp;
(xi) SEQ ID NO: 26, wherein X at position 170 of SEQ ID NO: 26 is Ser or Cys;
(xii) both (x) and (xi);
(xiii) SEQ ID NO: 27;
(xiv) SEQ ID NO: 28;
(xv) SEQ ID NO: 29;
(xvi) SEQ ID NO: 30;
(xvii) both (xiii) and (xiv); or
(xviii) both (xv) and (xvi).

10-11. (canceled).

12. An isolated or purified protein comprising at least one of the polypeptides of claim 8.

13. The isolated or purified protein according to claim 12, comprising:
(a) a first polypeptide chain comprising the amino acid sequences of SEQ ID NOs: 1-3;
(b) a second polypeptide chain comprising the amino acid sequences of SEQ ID NOs: 4-6; or
(c) both (a) and (b).

14. The isolated or purified protein according to claim 13, wherein:
(a) the first polypeptide chain comprises the amino acid sequence of SEQ ID NO: 7;
(b) the second polypeptide chain comprises the amino acid sequence of SEQ ID NO: 8;
(c) the first polypeptide chain comprises the amino acid sequence of SEQ ID NO: 9;
(d) the second polypeptide chain comprises the amino acid sequence of SEQ ID NO: 10;
(e) both (a) and (b); (f) both (c) and (d); (g) the first polypeptide chain comprises the amino acid sequence of SEQ ID NO: 23, wherein:
(i) X at position 181 of SEQ ID NO: 23 is Thr or Cys;
(ii) X at position 245 of SEQ ID NO: 23 is Ser, Ala, Val, Leu, Ile, Pro, Phe, Met, or Trp;
(iii) X at position 247 of SEQ ID NO: 23 is Met, Ala, Val, Leu, Ile, Pro, Phe, or Trp; and
(iv) X at position 248 of SEQ ID NO: 23 is Gly, Ala, Val, Leu, Ile, Pro, Phe, Met, or Trp;
(h) the second polypeptide chain comprises the amino acid sequence of SEQ ID NO: 24, wherein X at position 188 of SEQ ID NO: 24 is Ser or Cys;
(i) both (g) and (h);
(j) the first polypeptide chain comprises the amino acid sequence of SEQ ID NO: 25, wherein:
(i) X at position 161 of SEQ ID NO: 25 is Thr or Cys;
(ii) X at position 225 of SEQ ID NO: 25 is Ser, Ala, Val, Leu, Ile, Pro, Phe, Met, or Trp;
(iii) X at position 227 of SEQ ID NO: 25 is Met, Ala, Val, Leu, Ile, Pro, Phe, or Trp; and
(iv) X at position 228 of SEQ ID NO: 25 is Gly, Ala, Val, Leu, Ile, Pro, Phe, Met, or Trp;
(k) the second polypeptide chain comprises the amino acid sequence of SEQ ID NO: 26, wherein X at position 170 of SEQ ID NO: 26 is Ser or Cys;
(l) both (j) and (k);
(m) the first polypeptide chain comprises the amino acid sequence of SEQ ID NO: 27;
(n) the second polypeptide chain comprises the amino acid sequence of SEQ ID NO: 28;
(o) the first polypeptide chain comprises the amino acid sequence of SEQ ID NO: 29;
(p) the second polypeptide chain comprises the amino acid sequence of SEQ ID NO: 30;
(q) both (m) and (n); or
(r) both (o) and (p).

15-16. (canceled).

17. A bispecific engager TCR fusion protein comprising (i) the TCR according to claim 1 and (ii) an anti-CD3 effector.

18. An isolated or purified nucleic acid comprising a nucleotide sequence encoding the TCR according to claim 1.

19. An isolated or purified nucleic acid comprising, from 5′ to 3′, a first nucleic acid sequence and a second nucleotide sequence, wherein the first and second nucleotide sequence, respectively, encode the amino sequences of SEQ ID NOs: 7 and 8; 8and 7; 9 and 10; 10 and 9; 23 and 24; 24 and 23; 25 and 26; 26 and 25; 27 and 28; 28 and 27; 29 and 30; or 30 and 29.

20-21. (canceled).

22. A recombinant expression vector comprising the nucleic acid according to claim 18.

23-25. (canceled).

26. A method of producing a host cell expressing a TCR that has antigenic specificity for the peptide of VVVGAVGVGK (SEQ ID NO: 33), the method comprising contacting a cell with the vector according to claim 22 under conditions that allow introduction of the vector into the cell.

27. An isolated or purified host cell comprising the nucleic acid according to claim 18.

28-29. (canceled).

30. An isolated or purified population of cells comprising the host cell according to claim 27.

31. A method of producing a TCR, the method comprising culturing the host cell according to claim 27 so that the TCR is produced.

32. A pharmaceutical composition comprising (a) the TCR according to claim 1 and (b) a pharmaceutically acceptable carrier.

33. A method of detecting the presence of cancer in mammal, the method comprising:
(a) contacting a sample comprising cells of the cancer with the TCR according to claim 1, thereby forming a complex; and
(b) detecting the complex,
wherein detection of the complex is indicative of the presence of cancer in the mammal.

34-40. (canceled).

41. A method of inducing an immune response against cancer in a mammal, the method comprising administering to the mammal the TCR according to claim 1 in an amount effective to induce the immune response against cancer in the mammal.

42. A method of treating or preventing cancer in a mammal, the method comprising administering to the mammal the TCR according to claim 1 in an amount effective to treat or prevent cancer in the mammal.


SEQUENCE LISTING (34 sequences)

SEQ ID NO: 1 - protein, 7 aa, Mus musculus
DRNFQYF

SEQ ID NO: 2 - protein, 6 aa, Mus musculus
IFSDGD

SEQ ID NO: 3 - protein, 13 aa, Mus musculus
AARPGTQVVGQLT

SEQ ID NO: 4 - protein, 6 aa, Mus musculus
GKSSPN

SEQ ID NO: 5 - protein, 5 aa, Mus musculus
SITVG

SEQ ID NO: 6 - protein, 12 aa, Mus musculus
AWRDRPSSYEQY

SEQ ID NO: 7 - protein, 113 aa, Mus musculus
QQKVQQSPESLSVPEGGMASLNCTSSDRNFQYFWWYRQHSGEGPKALMSIFSDGDKKEGRFTAHLNKASLHVSLHIRDSQPSDSALYFCAARPGTQVVGQLTFGRGTRLQVYA

SEQ ID NO: 8 - protein, 113 aa, Mus musculus
AQTIHQWPVAEIKAVGSPLSLGCTIKGKSSPNLYWYWQATGGTLQQLFYSITVGQVESVVQLNLSASRPKDDQFILSTEKLLLSHSGFYLCAWRDRPSSYEQYFGPGTRLTVL

SEQ ID NO: 9 - protein, 133 aa, Mus musculus
MKSLSVLLVVLWLQLNCVRSQQKVQQSPESLSVPEGGMASLNCTSSDRNFQYFWWYRQHSGEGPKALMSIFSDGDKKEGRFTAHLNKASLHVSLHIRDSQPSDSALYFCAARPGTQVVGQLTFGRGTRLQVYA

SEQ ID NO: 10 - protein, 131 aa, Mus musculus
MALYSLLAFLLGMFLGVSAQTIHQWPVAEIKAVGSPLSLGCTIKGKSSPNLYWYWQATGGTLQQLFYSITVGQVESVVQLNLSASRPKDDQFILSTEKLLLSHSGFYLCAWRDRPSSYEQYFGPGTRLTVL

SEQ ID NO: 11 - protein, 189 aa, Homo sapiens [sequence omitted]

SEQ ID NO: 12 - protein, 188 aa, Homo sapiens [sequence omitted]

SEQ ID NO: 13 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 14 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 15 - protein, 189 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 16 - protein, 188 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 17 - protein, 189 aa, Homo sapiens [sequence omitted]

SEQ ID NO: 18 - protein, 189 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 19 - protein, 137 aa, synthetic construct
  REGION 1..137: note = Synthetic
  VARIANT 48: note = X at position 48 is T or C
  VARIANT 112: note = X at position 112 is S, A, V, L, I, P, F, M, or W
  VARIANT 114: note = X at position 114 is M, A, V, L, I, P, F, or W
  VARIANT 115: note = X at position 115 is G, A, V, L, I, P, F, M, or W
NIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 20 - protein, 173 aa, synthetic construct
  REGION 1..173: note = Synthetic
  VARIANT 57: note = X at position 57 is S or C
EDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 21 - protein, 137 aa, Mus musculus
NIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 22 - protein, 173 aa, Mus musculus
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 23 - protein, 270 aa, synthetic construct
  REGION 1..270: note = Synthetic
  VARIANT 181: note = X at position 181 is T or C
  VARIANT 245: note = X at position 245 is S, A, V, L, I, P, F, M, or W
  VARIANT 247: note = X at position 247 is M, A, V, L, I, P, F, or W
  VARIANT 248: note = X at position 248 is G, A, V, L, I, P, F, M, or W
MKSLSVLLVVLWLQLNCVRSQQKVQQSPESLSVPEGGMASLNCTSSDRNFQYFWWYRQHSGEGPKALMSIFSDGDKKEGRFTAHLNKASLHVSLHIRDSQPSDSALYFCAARPGTQVVGQLTFGRGTRLQVYANIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 24 - protein, 304 aa, synthetic construct
  REGION 1..304: note = Synthetic
  VARIANT 188: note = X at position 188 is S or C
MALYSLLAFLLGMFLGVSAQTIHQWPVAEIKAVGSPLSLGCTIKGKSSPNLYWYWQATGGTLQQLFYSITVGQVESVVQLNLSASRPKDDQFILSTEKLLLSHSGFYLCAWRDRPSSYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 25 - protein, 250 aa, synthetic construct
  REGION 1..250: note = Synthetic
  VARIANT 161: note = X at position 161 is T or C
  VARIANT 225: note = X at position 225 is S, A, V, L, I, P, F, M, or W
  VARIANT 227: note = X at position 227 is M, A, V, L, I, P, F, or W
  VARIANT 228: note = X at position 228 is G, A, V, L, I, P, F, M, or W
QQKVQQSPESLSVPEGGMASLNCTSSDRNFQYFWWYRQHSGEGPKALMSIFSDGDKKEGRFTAHLNKASLHVSLHIRDSQPSDSALYFCAARPGTQVVGQLTFGRGTRLQVYANIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 26 - protein, 286 aa, synthetic construct
  REGION 1..286: note = Synthetic
  VARIANT 170: note = X at position 170 is S or C
AQTIHQWPVAEIKAVGSPLSLGCTIKGKSSPNLYWYWQATGGTLQQLFYSITVGQVESVVQLNLSASRPKDDQFILSTEKLLLSHSGFYLCAWRDRPSSYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 27 - protein, 270 aa, Mus musculus
MKSLSVLLVVLWLQLNCVRSQQKVQQSPESLSVPEGGMASLNCTSSDRNFQYFWWYRQHSGEGPKALMSIFSDGDKKEGRFTAHLNKASLHVSLHIRDSQPSDSALYFCAARPGTQVVGQLTFGRGTRLQVYANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 28 - protein, 304 aa, Mus musculus
MALYSLLAFLLGMFLGVSAQTIHQWPVAEIKAVGSPLSLGCTIKGKSSPNLYWYWQATGGTLQQLFYSITVGQVESVVQLNLSASRPKDDQFILSTEKLLLSHSGFYLCAWRDRPSSYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 29 - protein, 250 aa, Mus musculus
QQKVQQSPESLSVPEGGMASLNCTSSDRNFQYFWWYRQHSGEGPKALMSIFSDGDKKEGRFTAHLNKASLHVSLHIRDSQPSDSALYFCAARPGTQVVGQLTFGRGTRLQVYANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 30 - protein, 286 aa, Mus musculus
AQTIHQWPVAEIKAVGSPLSLGCTIKGKSSPNLYWYWQATGGTLQQLFYSITVGQVESVVQLNLSASRPKDDQFILSTEKLLLSHSGFYLCAWRDRPSSYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 31 - protein, 27 aa, synthetic construct
  REGION 1..27: note = Synthetic
RAKRSGSGATNFSLLKQAGDVEENPGP

SEQ ID NO: 32 - protein, 601 aa, synthetic construct
  REGION 1..601: note = Synthetic
MALYSLLAFLLGMFLGVSAQTIHQWPVAEIKAVGSPLSLGCTIKGKSSPNLYWYWQATGGTLQQLFYSITVGQVESVVQLNLSASRPKDDQFILSTEKLLLSHSGFYLCAWRDRPSSYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNSRAKRSGSGATNFSLLKQAGDVEENPGPMKSLSVLLVVLWLQLNCVRSQQKVQQSPESLSVPEGGMASLNCTSSDRNFQYFWWYRQHSGEGPKALMSIFSDGDKKEGRFTAHLNKASLHVSLHIRDSQPSDSALYFCAARPGTQVVGQLTFGRGTRLQVYANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 33 - protein, 10 aa, Homo sapiens
VVVGAVGVGK

SEQ ID NO: 34 - protein, 10 aa, Homo sapiens
VVVGAGGVGK